(19) United States
(12) Patent Application Publication
(10) Pub. No.: US 2012/0003329 A1
(43) Pub. Date: Jan. 5, 2012

(54) Use of A2B Adenosine Receptor Antagonists for Treating Pulmonary Hypertension

(75) Inventors: Luiz Belardinelli, Palo Alto, CA (US); Dewan Zeng, Palo Alto, CA (US); Hongyan Zhong, Mountain View, CA (US)

(73) Assignee: Gilead Sciences, Inc.

(21) Appl. No.: 13/172,609

(22) Filed: Jun. 29, 2011

Related U.S. Application Data
(60) Provisional application No. 61/360,289, filed on Jun. 30, 2010.

(51) Int. Cl. A61K 31/522; C12N 5/071; A61P 9/00; A61P 11/00; A61K 33/00; A61P 9/12

(52) U.S. Cl. 424/718; 514/263.2; 435/375

(57) ABSTRACT

This disclosure relates generally to treating patients having pulmonary hypertension, or symptoms associated therewith, by administering a therapeutically effective amount of an A_{2B} receptor antagonist to the patient.

### CROSS REFERENCE TO RELATED APPLICATIONS

This application claims the benefit under 35 U.S.C. §119(e) of U.S. Provisional Application Ser. No. 61/360,289 filed Jun. 30, 2010, which is incorporated by reference in its entirety.

### FIELD OF THE DISCLOSURE

The disclosure is directed to methods of treating pulmonary hypertension in patients in need thereof by administering a therapeutically effective amount of an A_{2B} adenosine receptor antagonist.

### STATE OF THE ART

Pulmonary hypertension (PH) was initially classified by the World Health Organization (WHO), in 1973, as primary (idiopathic) or secondary, depending on the presence or absence of identificable causes for risk factors. The classification has gone through a series of changes. The current classification was adopted during the 4^{th} World Symposium on Pulmonary Hypertension held in 2008 in Dana Point, Calif. This new classification includes five groups for pulmonary hypertension:

Group 1: Pulmonary arterial hypertension (PAH);

Group 1′: Pulmonary veno-occlusive disease (PVOD) and/or pulmonary capillary hemangiomatosis (PCH);

Group 2: Pulmonary hypertension owing to left heart disease;

Group 3: Pulmonary hypertension owing to lung diseases and/or hypoxia;

Group 4: Chronic thromboembolic pulmonary hypertension (CTEPH); and

Group 5: Pulmonary hypertension with unclear multifactorial mechanisms. See, for example, Simonneau et al., J Am Coll Cardio, 54(1):543-54 (2009).

Pulmonary arterial hypertension (PAH), Group I of PH, is a serious, progressive, and life-threatening disease of the pulmonary vasculature, characterized by profound vasoconstriction and an abnormal proliferation of cells in the walls of the pulmonary arteries. This abnormal proliferation leads to severe constriction of the blood vessels in the lungs and, as a corollary, to very high pulmonary arterial pressures. These pressures make it difficult for the heart to pump adequate amounts of blood through the lungs for oxygenation. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have poor prognosis and, equally, a compromised quality of life, with a mean life expectancy of 2 to 5 years from the time of diagnosis if untreated.

Group 3 of PH is often associated with underlying chronic lung diseases such as chronic obstructive pulmonary disease (COPD) and pulmonary fibrosis. A predominant cause of Group 3 is alveolar hypoxia as a result of lung disease, impaired control of breathing, or residence at high altitude. This group includes chronic bronchiectasis, cystic fibrosis, and a newly identified syndrome characterized by the combination of pulmonary fibrosis, mainly of the lower zones of the lung, and emphysema, mainly of the upper zones of the lung. There is currently no approved medical therapy for patients with Group 3 pulmonary hypertension.

A variety of factors contribute to the pathogenesis of PH including proliferation of pulmonary cells which can contribute to vascular remodeling (i.e., hyperplasia). For example, pulmonary vascular remodeling occurs primarily by proliferation of arterial endothelial cells and smooth muscle cells of patients with PH. Steiner, et al., Interleukin-6 overexpression induces pulmonary hypertension, Circ. Res., available at http://circres.ahajournals.org (2009). Further, it has been found that PH may rise from the hyperproliferation of pulmonary arterial smooth cells and pulmonary endothelial cells. Id. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

In addition to the proliferation of pulmonary cells, altered expression of cytokines, growth factors, and chemokines may be found in the serum and/or lungs of PH patients. These altered expressions indicate a possible inflammatory mechanism or mediation in the pathogenesis of the disease. For example, it has been demonstrated that growth factor endotheline-1 (ET-1) and inflammatory cytokine interleukin (IL-6) is elevated in serum and lungs of PH patients. A. Giaid, et al., “Expression of endothelin-1 in the lungs of patients with pulmonary hypertension” N. Engl. J. Med., 329(26):1967-8 (1993) and Steiner, et al. (2009).

To date, a direct correlation between reduction in elevated ET-1 (or IL-6) levels by inhibiting the A_{2B} receptor in the pathogenesis of PH has not been made. Rather, the art has shown various methods for increasing ET-1 and IL-6, including activation of the A_{2B} adenosine receptor, as part of other disease modalities. For example, it is known in the art that activation of the A_{2B} receptor in bronchial smooth muscle cells and fibroblasts increases IL-6 release. Zhong, et al. “A_{2B} adenosine receptors increase cytokine release by bronchial smooth muscle cells,” Am. J. Resp. Cell. Mol., 30:118-125 (2004) and Zhong et al., “Synergy between A_{2B} Adenosine Receptors and Hypoxia in activating human lung fibroblasts,” Am. J. Respir. Cell Mol. Biol. 32:2-8 (2005). However, the bronchial tissue inflammation and fibroblast differentiation associated with stimulation of the A_{2B} receptor antagonist was only described as it relates to the pathogenesis of asthma. Therefore, there remains a need in the art to provide novel methods of treating PAH, including the vascular remodeling component, the proliferation component, and the inflammatory component of the disease.

### SUMMARY

This disclosure is directed to the surprising and unexpected discovery that a patient suffering from pulmonary hypertension may be treated using an A_{2B} adenosine receptor antagonist. It is contemplated that the hyperproliferation, vascular remodeling, and elevated levels of cytokines and chemokines associated with pulmonary hypertension patients is reduced by the A_{2B} adenosine receptor antagonist thereby treating the disease and/or the symptoms associated therewith.

It is also surprising to find that the effect of A_{2B} adenosine receptor antagonists to prevent and treat pulmonary hypertension is achieved by multiple mechanisms, including but not limited to through endothelial cells, smooth muscle cells, inflammatory cells) and multiple mediators, including but not limited to IL-6, IL-8, endothelin, thromboxane, collagen degradation products and extracellular matrix proteins. It is therefore contemplated that A_{2B} adenosine receptor antagonists are much more efficacious in the treatment of pulmonary hypertension, by virtue of these multiple mechanism and multiple mediators, compared to agents that target a single pathway, such as endothelin antagonists or phosphodiesterase inhibitors.

It has been discovered that A_{2B} receptors are expressed at high levels in human pulmonary arterial endothelial cells (HPAEC) and human pulmonary smooth muscle cells (HPASM). Moreover, it has been discovered that vascular wall thickening, one form of remodeling seen in pulmonary hypertension patients, is reduced by administration of such antagonists. Also reduced is the expression of collagen, other extracellular matrix proteins, and extracellular matrix enzymes in human pulmonary arterial smooth muscle cells (HPASM) associated with tissue remodeling. Further, proliferation and migration of HPASM, cells that are associated with vascular remodeling in PAH patients, are reduced by the antagonist. Still further, it has now been found that administration of an A_{2B} adenosine receptor antagonist reduces the production of ET-1 which is induced by activating the receptor in HPAEC. By reducing the ET-1, it is contemplated that the proliferation of the HPASM associated with pulmonary hypertension is also reduced. All of these findings suggest that pulmonary hypertension in a patient may be effectively treated by administration of an A_{2B} adenosine receptor antagonist. It is also contemplated that by treatment of the pulmonary hypertension, the right ventricle function is improved.

In a preclinical model of pulmonary hypertension owing to lung diseases (Group 3 of PH) the antagonist was shown to reduce vasculopathy and right ventricular systolic pressure (RVSP), to improve pulmonary vascular remodeling, and to increase oxygen saturation and improve lung functions.

In light of the above and in one of its method aspects, the disclosure is directed to a method for treating pulmonary hypertension to a patient in need thereof a therapeutically effective amount of an A_{2B} adenosine receptor antagonist. In one aspect, the pulmonary hypertension is one or more selected from Group 1, 1′, 2, 3, 4 or 5 pulmonary hypertension. In one aspect, the pulmonary hypertension is pulmonary arterial hypertension (PAH) or Group 1 of pulmonary hypertension. In another aspect, the pulmonary hypertension is pulmonary hypertension owing to lung diseases and/or hypoxia, or Group 3 of lung diseases and/or hypoxia.

In one embodiment, the A_{2B} adenosine receptor antagonist is a 8-cyclic xanthine derivative. In another embodiment, the A_{2B} adenosine receptor antagonist is a compound of Formula I or II:

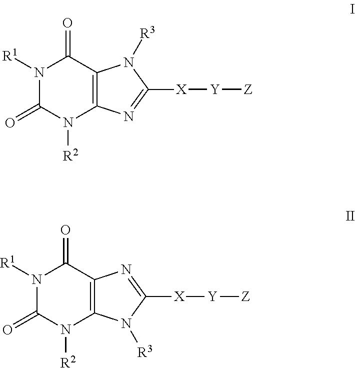

wherein:
R¹ and R² are independently chosen from hydrogen, optionally substituted alkyl, or a group -D-E, in which D is a covalent bond or alkylene, and E is optionally substituted alkoxy, optionally substituted cycloalkyl, optionally substituted aryl, optionally substituted heteroaryl, optionally substituted heterocyclyl, optionally substituted alkenyl or optionally substituted alkynyl, with the proviso that when D is a covalent bond E cannot be alkoxy;
R³ is hydrogen, optionally substituted alkyl or optionally substituted cycloalkyl;
X is optionally substituted arylene or optionally substituted heteroarylene;
Y is a covalent bond or alkylene in which one carbon atom can be optionally replaced by —O—, —S—, or —NH—, and is optionally substituted by hydroxy, alkoxy, optionally substituted amino, or —COR¹⁶, in which R¹⁶ is hydroxy, alkoxy or amino;
with the proviso that when the optional substitution is hydroxy or amino it cannot be adjacent to a heteroatom; and
Z is optionally substituted monocyclic aryl or optionally substituted monocyclic heteroaryl; or
Z is hydrogen when X is optionally substituted heteroarylene and Y is a covalent bond;
with the proviso that when X is optionally substituted arylene, Z is optionally substituted monocyclic heteroaryl
or a pharmaceutically acceptable salt, tautomer, isomer, a mixture of isomers, or prodrug thereof.

In another embodiment, the A_{2B} adenosine receptor antagonist is a compound selected from the group consisting of:
1-propyl-8-(1-{[3-(trifluoromethyl)phenyl]-methyl}pyrazol-4-yl)-1,3,7-trihydropurine-2,6-dione;
1-propyl-8-[1-benzylpyrazol-4-yl]-1,3,7-trihydropurine-2,6-dione;
1-butyl-8-(1-{[3-fluorophenyl]methyl}pyrazol-4-yl)-1,3,7-trihydropurine-2,6-dione;
1-propyl-8-[1-(phenylethyl)pyrazol-4-yl]-1,3,7-trihydropurine-2,6-dione;
8-(1-{[5-(4-chlorophenyl) (1,2,4-oxadiazol-3-yl)]methyl}pyrazol-4-yl)-1-propyl-1,3,7-trihydropurine-2,6-dione;
8-(1-{[5-(4-chlorophenyl)(1,2,4-oxadiazol-3-yl)]methyl}pyrazol-4-yl)-1-butyl-1,3,7-trihydropurine-2,6-dione;
1,3-dipropyl-8-pyrazol-4-yl-1,3,7-trihydropurine-2,6-dione;
1-methyl-3-sec-butyl-8-pyrazol-4-yl-1,3,7-trihydropurine-2,6-dione;
1-cyclopropylmethyl-3-methyl-8-{1-[(3-trifluoromethylphenyl)methyl]pyrazol-4-yl}-1,3,7-trihydropurine-2,6-dione;
1,3-dimethyl-8-{1-[(3-fluorophenyl)methyl]pyrazol-4-yl}-1,3,7-trihydropurine-2,6-dione;
3-methyl-1-propyl-8-{1-[(3-trifluoromethylphenyl)methyl]pyrazol-4-yl}-1,3,7-trihydropurine-2,6-dione;
3-ethyl-1-propyl-8-{1-[(3-trifluoromethylphenyl)methyl]pyrazol-4-yl}-1,3,7-trihydropurine-2,6-dione;
1,3-dipropyl-8-(1-{[3-(trifluoromethyl)phenyl]methyl}pyrazol-4-yl)-1,3,7-trihydropurine-2,6-dione;
1,3-dipropyl-8-{1-[(3-fluorophenyl)methyl]pyrazol-4-yl}-1,3,7-trihydropurine-2,6-dione;
1-ethyl-3-methyl-8-{1-[(3-fluorophenyl)methyl]pyrazol-4-yl}-1,3,7-trihydropurine-2,6-dione;
1,3-dipropyl-8-{1-[(2-methoxyphenyl)methyl]pyrazol-4-yl}-1,3,7-trihydropurine-2,6-dione;
1,3-dipropyl-8-(1-{[3-(trifluoromethyl)-phenyl]ethyl}pyrazol-4-yl)-1,3,7-trihydropurine-2,6-dione;
1,3-dipropyl-8-{1-[(4-carboxyphenyl)methyl]pyrazol-4-yl}-1,3,7-trihydropurine-2,6-dione;
2-[4-(2,6-dioxo-1,3-dipropyl(1,3,7-trihydropurin-8-yl))pyrazolyl]-2-phenylacetic acid;
8-{4-[5-(2-methoxyphenyl)-[1,2,4]oxadiazol-3-ylmethoxy]phenyl}-1,3-dipropyl-1,3,7-trihydropurine-2,6-dione;
8-{4-[5-(3-methoxyphenyl)-[1,2,4]oxadiazol-3-ylmethoxy]phenyl}-1,3-dipropyl-1,3,7-trihydropurine-2,6-dione;
8-{4-[5-(4-fluorophenyl)-[1,2,4]oxadiazol-3-ylmethoxy]phenyl}-1,3-dipropyl-1,3,7-trihydropurine-2,6-dione:
1-(cyclopropylmethyl)-8-[1-(2-pyridylmethyl)pyrazol-4-yl]-1,3,7-trihydropurine-2,6-dione;
1-n-butyl-8-[1-(6-trifluoromethylpyridin-3-ylmethyl)pyrazol-4-yl]-1,3,7-trihydropurine-2,6-dione;
8-(1-{[3-(4-chlorophenyl)(1,2,4-oxadiazol-5-yl)]methyl}pyrazol-4-yl)-1,3-dipropyl-1,3,7-trihydropurine-2,6-dione;
1,3-dipropyl-8-[1-({5-[4-(trifluoromethyl)phenyl]isoxazol-3-yl}methyl)pyrazol-4-yl]-1,3,7-trihydropurine-2,6-dione;
1,3-dipropyl-8-[1-(2-pyridylmethyl)pyrazol-4-yl]-1,3,7-trihydropurine-2,6-dione;
3-{[4-(2,6-dioxo-1,3-dipropyl-1,3,7-trihydropurin-8-yl)pyrazolyl]methyl}benzoic acid;
1,3-dipropyl-8-(1-{[6-(trifluoromethyl)(3-pyridyl)]methyl}pyrazol-4-yl)-1,3,7-trihydropurine-2,6-dione;
1,3-dipropyl-8-{1-[(3-(1H-1,2,3,4-tetraazol-5-yl)phenyl)methyl]pyrazol-4-yl}-1,3,7-trihydropurine-2,6-dione;
6-{[4-(2,6-dioxo-1,3-dipropyl-1,3,7-trihydropurin-8-yl)pyrazolyl]methyl}pyridine-2-carboxylic acid;
3-ethyl-1-propyl-8-[1-(2-pyridylmethyl)pyrazol-4-yl]-1,3,7-trihydropurine-2,6-dione;
8-(1-{[5-(4-chlorophenyl)isoxazol-3-yl]methyl}pyrazol-4-yl)-3-ethyl-1-propyl-1,3,7-trihydropurine-2,6-dione;
8-(1-{[3-(4-chlorophenyl)(1,2,4-oxadiazol-5-yl)]methyl}pyrazol-4-yl)-3-ethyl-1-propyl-1,3,7-trihydropurine-2,6-dione;
3-ethyl-1-propyl-8-(1-{[6-(trifluoromethyl)(3-pyridyl)]methyl}pyrazol-4-yl)-1,3,7-trihydropurine-2,6-dione;
1-(cyclopropylmethyl)-3-ethyl-8-(1-{[6-(trifluoromethyl)(3-pyridyl)]methyl}pyrazol-4-yl)-1,3,7-trihydropurine-2,6-dione; and
3-ethyl-1-(2-methylpropyl)-8-(1-{[6-(trifluoromethyl)(3-pyridyl)]methyl}pyrazol-4-yl)-1,3,7-trihydropurine-2,6-dione
or a pharmaceutically acceptable salt, tautomer, isomer, a mixture of isomers, or prodrug thereof.

In another embodiment of the disclosure, the A_{2B} adenosine receptor antagonist is a prodrug of Formula III having the formula:

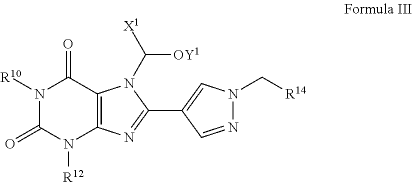

wherein:
R¹⁰ and R¹² are independently lower alkyl;
R¹⁴ is optionally substituted phenyl;
X¹ is hydrogen or methyl; and
Y¹ is —C(O)R¹⁷, in which R¹⁷ is independently optionally substituted lower alkyl, optionally substituted aryl, or optionally substituted heteroaryl; or
Y¹ is —P(O)(OR¹⁵)₂, in which R¹⁵ is hydrogen or lower alkyl optionally substituted by phenyl or heteroaryl;
and the pharmaceutically acceptable salts thereof.

Compounds or prodrugs of Formula III include, but are not limited to, the following compounds:
[3-ethyl-2,6-dioxo-1-propyl-8-(1-{[3-(trifluoromethyl)phenyl]methyl}pyrazol-4-yl)-1,3,7-trihydropurin-7-yl]methyl acetate;
[3-ethyl-2,6-dioxo-1-propyl-8-(1-{[3-(trifluoromethyl)phenyl]methyl}pyrazol-4-yl)-1,3,7-trihydropurin-7-yl]methyl 2,2-dimethylpropanoate;
[3-ethyl-2,6-dioxo-1-propyl-8-(1-{[3-(trifluoromethyl)phenyl]methyl}pyrazol-4-yl)-1,3,7-trihydropurin-7-yl]methyl butanoate; and
[3-ethyl-2,6-dioxo-1-propyl-8-(1-{[3-(trifluoromethyl)phenyl]methyl}-pyrazol-4-yl)(1,3,7-trihydropurin-7-yl)]methyl dihydrogen phosphate
or a pharmaceutically acceptable salt thereof.

In still yet another embodiment of the disclosure, the A_{2B} adenosine receptor antagonist is 3-ethyl-1-propyl-8-(1-(3-(trifluoromethyl)benzyl)-1H-pyrazol-4-yl)-1H-purine-2,6(3H,7H)-dione or 3-ethyl-1-propyl-8-(1-((3-(trifluoromethyl)phenyl)methyl)pyrazol-4-yl)-1,3,7-trihydropurine-2,6-dione (referred to throughout as “Compound A” or “Comp A”), having the following chemical formula:

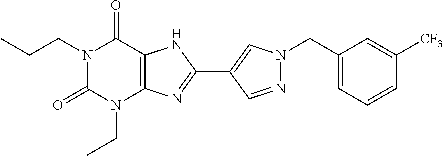

or a pharmaceutically acceptable salt, tautomer, isomer, a mixture of isomers, or prodrug thereof, where the term prodrug is as defined in Formula III.

In another of its method aspects, this disclosure is directed to a method of inhibiting overexpression of collagen, other extracellular matrix proteins, and extracellular matrix enzymes in human pulmonary arterial smooth muscle cells (HPASM) which method comprises contacting these cells with an effective amount of an A_{2B} adenosine receptor antagonist.

In yet another of its method aspects, this disclosure is directed to a method of reducing IL-6, IL-8, G-CSF, and/or thromboxane release from pulmonary arterial smooth muscle cells which method comprises contacting these cells with an effective amount of an A_{2B} adenosine receptor antagonist.

In another aspect, this disclosure is directed to a method of reducing IL-8 and/or ET-1 expression in pulmonary arterial endothelial cells which method comprises contacting these cells with an effective amount of an A_{2B} adenosine receptor antagonist.

Still in another aspect, this disclosure is directed to a method of inhibiting proliferation or migration of a pulmonary arterial smooth muscle cell which method comprises contacting the cell with an effective amount of an A_{2B} adenosine receptor antagonist.

In another aspect, this disclosure is directed to a method of inhibiting vascular wall thickening in a patient in need thereof, which comprises administering to the patient a therapeutically effective amount of an A_{2B} adenosine receptor antagonist.

In a further aspect, this disclosure is directed to a method of decreasing right ventricular systolic pressure (RVSP) and/or right ventricular hypertrophy in a patient in need thereof, which comprises administering to the patient a therapeutically effective amount of an A_{2B} adenosine receptor antagonist.

Further, in an aspect, this disclosure is directed to a method of improving lung function in a patient in need thereof, which comprises administering to the patient a therapeutically effective amount of an A_{2B} adenosine receptor antagonist.

### BRIEF DESCRIPTION OF THE DRAWINGS

The disclosure is best understood from the following detailed description when read in conjunction with the accompanying drawings. Included in the drawings are the following figures:

FIG. 1 illustrates mRNA expression of four subtypes of adenosine receptors (A₁, A_{2A}, A_{2B}, and A₃) on human pulmonary arterial endothelial cells (HPAEC) obtained using quantitative real-time RT-PCR as described in Example 3. As can be seen, A_{2B} expression was the highest amongst the four subtypes of adenosine receptors.

FIG. 2 illustrates mRNA expression of four subtypes of adenosine receptors (A₁, A_{2A}, A_{2B}, and A₃) on human pulmonary arterial smooth muscle cells (HPASM) obtained using quantitative real-time RT-PCR as described in Example 3. As can be seen, A_{2B} expression was the highest amongst the four subtypes of adenosine receptors.

FIG. 3A-C depict the differences in pulmonary histopathology in control mice (3A), adenosine deaminase (ADA)−/− mice (3B), and adenosine deaminase (ADA)−/− mice after treatment with an A_{2B} adenosine receptor antagonist, Compound A (“Comp A”) (3C). Procedures were as described in Example 13. As can be seen in 3C, vascular wall thickening caused by adenosine abundance was drastically reduced by treatment with the A_{2B} adenosine receptor antagonist.

FIG. 4A-I show the vascular changes in wild type and A_{2B} receptor knockout (KO) mice exposed to bleomycin. FIGS. 4A, 4D, and 4G show the distal arteries, proximal arteries, and preacinar pulmonary arteries, respectively, from wild type mice exposed to saline. FIGS. 4B, 4E and 4H show the distal arteries, proximal arteries, and preacinar pulmonary arteries, respectively, from wild type mice exposed to bleomycin. FIGS. 4C, 4F and 4I show the distal arteries, proximal arteries, and preacinar pulmonary arteries, respectively, from A_{2B} receptor KO mice exposed to bleomycin. Wild type mice exposed to bleomycin showed increased muscularity around the small distal pulmonary arteries and more proximal pulmonary arteries, suggesting that these mice had classical morphological features of PAH. The A_{2B} receptor KO mice exposed to bleomycin did not exhibit these vascular changes indicating that the A_{2B} receptor is involved in the pathogenesis of PH.

FIG. 5 presents the levels of chemokine, IL-8, as measured by ELISA, in HPAECs after the cells were incubated for 18 hours with control, NECA (N-ethylcarboxamide adenosine) at various concentrations (0.1 μM, 1 μM, and 10 μM), and NECA (10 μM) together with Compound A, an A_{2B} adenosine receptor antagonist, (100 nM). NECA dose-dependently increased the release of IL-8 and the NECA-induced release of IL-8 was inhibited by Compound A, suggesting that the activation of A_{2B} receptor induced the release of IL-8. Data was obtained according to the procedure described in Example 5. *, p<0.05 compared to control; #, p<0.05 compared to NECA (10 μM).

FIG. 6 presents the level of endothelin, ET-1, as measured by ELISA, in HPAECs after the cells were incubated for 18 hours with control, NECA at various concentrations (0.1 μM, 1 μM, and 10 μM), and NECA (10 μM) together with Compound A (100 nM). NECA dose-dependently increased the release of ET-1 and the NECA-induced release of ET-1 is inhibited by Compound A in HPAECs, suggesting that the activation of A_{2B} receptor induced the release of ET-1. Data was obtained according to the procedure described in Example 6. *, p<0.05 compared to control; #, p<0.05 compared to NECA (10 μM).

FIG. 7 presents the levels of inflammatory cytokine, IL-6, as measured by ELISA, in HPASMs after the cells were incubated for 18 hours with control, NECA at various concentrations (0.1 μM, 1 μM, and 10 μM), and NECA (10 μM) together with Compound A (100 nM). NECA dose-dependently increased the release of IL-6 and the NECA-induced release of IL-6 is inhibited by Compound A in HPASMs. Data was obtained according to the procedure in Example 7. *, p<0.05 compared to control; #, p<0.05 compared to NECA (10 μM).

FIG. 8 presents the levels of chemokine, IL-8, as measured by ELISA, in HPASMs after the cells were incubated for 18 hours with control, NECA at various concentrations (0.1 μM, 1 μM, and 10 μM), and NECA (10 μM) together with Compound A (100 nM). NECA dose-dependently increased the release of IL-8 and the NECA-induced release of IL-8 is inhibited by Compound A in HPASMs. Data was obtained according to the procedure in Example 7. *, p<0.05 compared to control; #, p<0.05 compared to NECA (10 μM).

FIG. 9 presents the levels of G-CSF as measured by ELISA, in HPASMs after the cells were incubated for 18 hours with control, NECA at various concentrations (0.1 μM, 1 μM, and 10 μM), and NECA (10 μM) together with Compound A (100 nM). NECA dose-dependently increased the release of G-CSF and the NECA-induced release of G-CSF is inhibited by Compound A in HPASMs. Data was obtained according to the procedure in Example 7.

FIG. 10 shows the rates of smooth muscle cell migration with HPASMs treated with vehicle medium, NECA (10 μM) medium, NECA (10 μM) and Compound A (100 nM) medium, or NECA (10 μM) medium and an anti-IL-6 antibody for 18 hours. Conditional media collected from HPASMs treated with vehicle, NECA (10 μM) or Compound A (100 nM) for 18 h were added to the lower wells of Boyden chamber assay systems as chemoattractants. HPASMs were allowed to migrate for 24 hrs. (A): NECA medium increased smooth muscle cell migration and the incease was inhibited by either Compound A or the anti-IL-6 antibody. (B): illustration of a proposed mechanism in which, through activating A_{2B} adenosine receptor, NECA activates smooth muscle which releases IL-6. The released IL-6 in turn enhances smooth muscle cell migration. *, p<0.05 compared to control; #, p<0.05 compared to NECA (10 μM).

FIG. 11 presents the levels of thromboxane B2, a potent arterial vasoconstrictor, as measured by ELISA, in HPASMs after the cells were incubated for 18 hours with control, NECA at various concentrations (0.1 μM, 1 μM, and 10 μM), and NECA (10 μM) together with Compound A (100 nM). NECA dose-dependently increased the release of thromboxane and the NECA-induced release of thromboxane B2 in HPASMs. Data was obtained according to the procedure in Example 9. *, p<0.05 compared to control; #, p<0.05 compared to NECA (10 μM).

FIG. 12A-C show the levels of expression of various collagen, extracellular matrix proteins, and extracellular matrix enzymes important in tissue remodeling after treatment with Compound A. Data was obtained according to the procedure in Example 10. As can be seen, activation of the A_{2B} receptor induced the release of some of these genes (A and B) but the induction was inhibited by Compound A (C).

FIG. 13A-B show the results of HPAECs that were treated with vehicle (control medium), NECA (10 μM, NECA medium) or NECA and Compound A (100 nM) for 18 hours. The cell supernatants (diluted 1:1 in SM serum-free medium) were used to incubate HPASMs for 18 hours according to Example 11. NECA-HPAEC medium increased cell number of HPASMs at 18 hours compared to control-HPAEC medium. (A) NECA itself did not increase proliferations of HPAECs (data not shown). This finding suggests that certain mediator induced by NECA and released from HPAEC may be able to promote proliferation of HPASM or prevent cell death of HPASM. (B): Treatment with both Compound A inhibited the NECA induced proliferation. Therefore, adenosine activated HPAECs are able to induce proliferation of the HPASMs, and this is mediated by A_{2B} receptors in HPAECs. *, p<0.05 compared to control; #, p<0.05 compared to NECA (10 μM).

FIG. 14 shows the NOTCH3 expression in HPASMs that were incubated with NECA (10 μM) or NECA (10 μM) and Compound A (100 nM) for 1.5 hours. NECA increased the expression of NOTCH3 and this effect of NECA was inhibited by Compound A. *, p<0.05 compared to control; #, p<0.05 compared to NECA (10 μM).

FIG. 15 illustrates the dosing schedule in Example 14.

FIG. 16. presents that both adenosine level and expression of A2BR are increased following bleomycin treatment. (A) Adenosine levels, measured by HPLC, from bronchoalveolar lavage fluid (BALF) of mice treated with PBS or bleomycin (BLM) and sacrificed on day 33. A_{2B}R (B) transcript levels from fresh frozen lungs of mice treated with PBS or BLM.

FIG. 17 presents pictures and charts showing increased pulmonary vascular muscularization following bleomycin exposure and the inhibitory effects of Compound A. Compound A (10 mg/kg/day) was administered in the diet, PBS and BLM groups were provided with a control diet. (A) Immunostaining for α-SMA to identify myofibroblasts (gray signal) in the parenchyma (upper panels) and the muscular wall of vessels (arrows and lower panels). Morphometric analysis was conducted to determine the extent of muscularization present in 5-7 vessels for each mouse in all treatment groups (B) and the number of muscularized vessels observed in 10 random micropictographs of the lung parenchyma of each mouse in all groups (C). Results are presented as mean±SEM, N=5-8 for all treatment groups. Significance levels: ***P<0.001 refers to comparisons between PBS and BLM treatment groups. Significance levels: #P<0.05, # # 0.001<P<0.01 refer to ANOVA comparisons between BLM and BLM+Compound A or BLM+A_{2B}R^{-/-} (Zhou et al. J Immunol 182:8037-46 (2009)).

FIG. 18 presents charts showing cardiovascular physiology after bleomycin treatment and the effects of Compound A. Antagonizing or knockout of A_{2B}R inhibits the increase in RVSP in mice treated with bleomycin. Compound A (10 mg/kg/day) was administered in the diet, PBS and BLM groups were provided with a control diet. Results are presented as mean±SEM, N=6-8 for all treatment groups. Significance levels: ***P<0.001 and **0.001<P<0.01 refer to comparisons between PBS and BLM treatment groups. Significance levels: # # # P<0.001 refer to ANOVA comparisons between BLM and BLM+Compound A or BLM+A_{2B}R^{-/-} treatment groups.

FIG. 19 presents peri-vascular fibrosis in the lung. Antagonizing or knockout of A_{2B}R inhibits belomycine-induced peri-vascular fibrosis in the lung. Representative histological sections stained with Masson's trichrome to reveal collagen fibers (gray signal) of mice treated with PBS, BLM, BLM+Compound A and BLM exposed A_{2B}R^{-/-} mice. The asterisk denotes the region where the fibrotic fibers are present.

FIG. 20 presents lung function measurements after bleomycin treatment and the effects of Compound A. Antagonizing or knockout of A_{2B}R improves pulmonary function in mice treated with bleomycin. (A) Dynamic resistance of the lungs, (B) tissue damping (resistance) parameters and (C) Quasi-static elastance reflecting the elastic recoil pressure on the lungs at a given volume. Measurements were performed using a Flexivent system in tracheotomized and anaesthetized mice. (D) Arterial oxygenation levels were determined in awake mice by pulse oximetry using the MouseOx system. Experimental groups included mice that were treated with PBS, PBS and Compound A, BLM, BLM and Compound A or A_{2B}R^{-/-} treated with BLM. Results are presented as mean±SEM, n=8-9 for all treatment groups. Significance levels: ***P<0.001 refers to comparisons between PBS and BLM treatment groups. Significance levels: # # #P<0.001, # # 0.001<P<0.01 and #P<0.05, refer to ANOVA comparisons between BLM and BLM+Compound A treatment groups.

FIG. 21 shows interleukin (IL)-6 levels after bleomycin treatment and the effects of Compound A. Antagonizing or knockout of A_{2B}R reduces bleomycin-induced IL-6 in BALF and plasma IL-6 protein levels in BALF (A) and plasma (B) collected on day 33 following treatment regimen and were determined using ELISA. Experimental groups included mice that were treated with PBS, PBS and Compound A, BLM, BLM and Compound A or A_{2B}R^{-/-} treated with BLM. Results are presented as mean±SEM, n=4-6 for all treatment groups. Significance levels: ***P<0.001 refers to comparisons between PBS and BLM treatment groups. Significance levels: # # #P<0.001 refer to ANOVA comparisons between BLM and BLM+Compound A treatment groups.

FIG. 22 presents plasma ET-1 level and ET-1 expression in the lung following treatment with bleomycin. Antagonizing or knockout of A_{2B}R inhibits bleomycin-induced plasma ET-1 and expression of ET-1 in pulmonary vessel wall. (A) Protein levels of ET-1 in plasma determined by ELISA. (B) Immunofluorescent staining for the ET-1 (light gray) from mice treated with PBS, BLM, BLM+Compound A and A_{2B}R^{-/-} mice treated with BLM. The arrows denote the location of the vessel wall.

### DETAILED DESCRIPTION

Prior to describing this disclosure in greater detail, the following terms will first be defined.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a thread” includes a plurality of threads.

### 1. DEFINITIONS

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

As stated above, the disclosure is directed to a method of treating pulmonary hypertension comprising administering to a patient in need thereof a therapeutically effective amount of an A_{2B adenosine receptor antagonist.}

The term “treatment” means any treatment of a disease in a patient including: (i) preventing the disease, that is causing the clinical symptoms not to develop; (ii) inhibiting the disease, that is, arresting the development of clinical symptoms; and/or (iii) relieving the disease, that is, causing the regression of clinical symptoms. By way of example only, treating may include improving right ventricular function and/or alleviating symptoms, including, but not limited to exertional dyspnea, fatigue, chest pain, and combinations thereof.

As used herein, the term “pulmonary hypertension” or “PH” refers to an increase in blood pressure in the pulmonary artery, pulmonary vein, or pulmonary capillaries. Detailed description and classification of pulmonary hypertension can be found, for instance, at Simonneau et al., J Am Coll Cardio, 54(1):S43-54 (2009) and throughout the text.

As used herein, the term “pulmonary arterial hypertension” or “PAH” is intended to include idiopathic PAH, familial PAH, pulmonary veno-occlusive disease (PVOD), pulmonary capillary hemangiomatosis (PCH), persistent pulmonary hypertension of the newborn, or PAH associated with another disease or condition, such as, but not limited to, collagen vascular disease, congenital systemic-to-pulmonary shunts (including Eisenmenger's syndrome), portal hypertension, HIV infection, drugs and toxins, thyroid disorders, glycogen storage disease, Gaucher disease, hereditary hemorrhagic telangiectasia, hemoglobinopathies, myeloproliferative disorders, or splenectomy.

The term “extracellular matrix protein” refers to a protein, or a gene encoding the protein, being part of the extracellular part of animal tissue that provides structural support to the animal cells in addition to performing various other functions. Examples of extracellular matrix protein includes, without limitation, collagen, elastin, fibronectin and laminin.

The term “extracellular matrix enzyme” refers to a protein, or a gene encoding the protein, that is involved in the breakdown of extracellular matrix in normal physiological processes, such as embryonic development, reproduction, and tissue remodeling, as well as in disease processes, such as arthritis and metastasis. Non-limiting examples include MMP1, MMP2, MMP3, MMPI, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28.

The term “collagen” refers to one or more proteins or genes encoding such proteins, which are in the form of elongated fibrils, mostly found in animal fibrous tissues such as tendon, ligament and skin. Non-limiting examples of collagen include COL1A1, COL1A2, COL2A1, COL3A1, COL4A1, COL4A2, COL4A3, COL4A4, COL4A5, COL4A6, COL5A1, COL5A2, COL5A3, COL6A1, COL6A2, COL6A3, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, EMID2, COL27A1, COL28A1 and COL29A1.

The term “patient” typically refers to a mammal, such as, for example, a human.

The term “therapeutically effective amount” refers to that amount of a compound, such as an A_{2B} adenosine receptor antagonist, that is sufficient to effect treatment, as defined above, when administered to a patient in need of such treatment. The therapeutically effective amount will vary depending upon the specific activity or delivery route of the agent being used, the severity of the patient's disease state, and the age, physical condition, existence of other disease states, and nutritional status of the patient. Additionally, other medication the patient may be receiving will affect the determination of the therapeutically effective amount of the therapeutic agent to administer.

The term “A_{2B} adenosine receptor” or “A_{2B} receptor” refers to a subtype of an adenosine receptor. Other subtypes include A₁, A_{2A} and A₃.

The term “A_{2B} adenosine receptor antagonist” or “A_{2B} receptor antagonist” refers to any compound, peptides, proteins (e.g., antibodies), siRNA that inhibits or otherwise modulates the expression or activity of the A_{2B} adenosine receptor. In one embodiment, the antagonist selectively inhibits the A_{2B} receptor over the other subtypes of adenosine receptor. In another embodiment the antagonist is partially selective for the A_{2B} receptor. Compounds that are putative antagonists may be screened using the procedure in Example 2. Examples of antagonists include, but not limited to, those discussed in the section below.

In one embodiment, the A_{2B} receptor antagonist is a compound having the chemical formula:

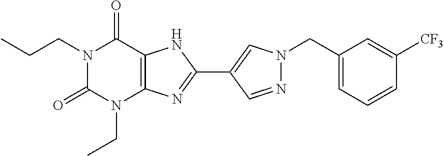

and the name 3-ethyl-1-propyl-8-(1-(3-(trifluoromethyl)benzyl)-1H-pyrazol-4-yl)-1H-purine-2,6(3H,7H)-dione or 3-ethyl-1-propyl-8-(1-((3-(trifluoromethyl)phenyl)methyl)pyrazol-4-yl)-1,3,7-trihydropurine-2,6-dione. It is sometimes referred to throughout as “Compound A” or “Comp A.” The compound is described in U.S. Pat. No. 6,825,349, which is hereby incorporated by reference in its entirety.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl group as defined above, having 1, 2, 3, 4 or 5 substituents, preferably 1 to 3 substituents, selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2; or

2) an alkyl group as defined above that is interrupted by 1-10 atoms independently chosen from oxygen, sulfur and NRₐ—, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR²⁰, in which R²⁰ is alkyl, aryl, or heteroaryl and n is 0, 1 or 2; or

3) an alkyl group as defined above that has both 1, 2, 3, 4 or 5 substituents as defined above and is also interrupted by 1-10 atoms as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents, preferably 1, 2, or 3 substituents, as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, preferably 1-10 carbon atoms, more preferably 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “lower alkylene” refers to a diradical of a branched or unbranched saturated hydrocarbon chain, preferably having from 1, 2, 3, 4, 5, or 6 carbon atoms.

The term “substituted alkylene” refers to:

(1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2; or

(2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocycyl, or groups selected from carbonyl, carboxyester, carboxyamide and sulfonyl; or

(3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “alkoxy” refers to the group R²¹—O—, where R²¹ is optionally substituted alkyl or optionally substituted cycloalkyl, or R²¹ is a group —Y¹¹—Z¹¹, in which Y¹¹ is optionally substituted alkylene and Z¹¹ is optionally substituted alkenyl, optionally substituted alkynyl; or optionally substituted cycloalkenyl, where alkyl, alkenyl, alkynyl, cycloalkyl and cycloalkenyl are as defined herein. Preferred alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio” refers to the group R²¹—S—, where R²¹ is as defined for alkoxy.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having 1-6, preferably 1, double bond (vinyl). Preferred alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or prop-1-yn-3-yl, —CH₂C≡CH), and the like. In the event that alkynyl is attached to nitrogen, the triple bond cannot be alpha to the nitrogen.

The term “substituted alkynyl” refers to an alkynyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “aminocarbonyl” refers to the group —C(O)NR²²R²² where each R²² is independently hydrogen, alkyl, aryl, heteroaryl, heterocyclyl or where both R¹² groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “alkoxycarbonylamino” refers to the group —NR³OC(O)OR³¹ where R³⁰ is hydrogen or alkyl and R³¹ is selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic.

The term “aminosulfonyl” refers to the group —SO₂NR³²R³³ where R³² and R³³ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R³² and R³³ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “azido” refers to the group N₃—.

The term “aminocarbonylamino” refers to the group —NR³⁴C(O)NR³⁵R³⁶ where R³⁴ is hydrogen or alkyl and R³⁵ and R³⁶ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R³⁵ and R³⁶ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “alkoxyamino” refers to the group —NR³⁷OR³⁸ where R³⁷ is hydrogen or alkyl and R³⁸ is selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic.

The term “acylamino” refers to the group —NR²³C(O)R²³ where each R²³ is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, or —S(O)ₙR²⁰, where R²⁰ is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl or anthryl). Preferred aryls include phenyl, naphthyl and the like.

The term “arylene” refers to a diradical of an aryl group as defined above. This term is exemplified by groups such as 1,4-phenylene, 1,3-phenylene, 1,2-phenylene, 1,4′-biphenylene, and the like.

Unless otherwise constrained by the definition for the aryl or arylene substituent, such aryl or arylene groups can optionally be substituted with from 1 to 5 substituents, preferably 1 to 3 substituents, selected from the group consisting of alkyl, alkenyl, alkynyl, alkoxy, cycloalkyl, cycloalkenyl, acyl, acylamino, aryloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group aryl-S—, where aryl is as defined above.

The term “amino” refers to the group —NH₂.

The term “substituted amino” refers to the group —NR²⁴R²⁴ where each R²⁴ is independently selected from the group consisting of hydrogen, alkyl, cycloalkyl, carboxyalkyl (for example, benzyloxycarbonyl), aryl, heteroaryl and heterocyclyl provided that both R¹⁴ groups are not hydrogen, or a group —Y¹²—Z¹², in which Y¹² is optionally substituted alkylene and Z¹² is alkenyl, cycloalkenyl, or alkynyl, Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “carboxyalkyl” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, or —S(O)ₙR²⁰, in which R²⁰ is alkyl, aryl, or heteroaryl and n is 0, 1 or 2.

The term “cycloalkyl” refers to carbocyclic groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

The term “halogen” or “halo” refers to fluoro, bromo, chloro, and iodo.

The term “acyl” denotes a group —C(O)R²⁵, in which R²⁵ is hydrogen, optionally substituted alkyl, optionally substituted cycloalkyl, optionally substituted heterocyclyl, optionally substituted aryl, and optionally substituted heteroaryl.

The term “heteroaryl” refers to a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-oxide and N-alkoxy-nitrogen derivatives containing heteroaryl compounds, for example pyridine-N-oxide derivatives.

The term “heteroarylene” refers to a diradical of a heteroaryl group as defined above. This term is exemplified by groups such as 2,5-imidazolene, 3,5-[1,2,4]oxadiazolene, 2,4-oxazolene, 1,4-pyrazolene, and the like. For example, 1,4-pyrazolene is:

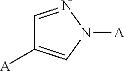

where A represents the point of attachment.

Unless otherwise constrained by the definition for the heteroaryl or heteroarylene substituent, such heteroaryl or heterarylene groups can be optionally substituted with 1 to 5 substituents, preferably 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “heteroaryloxy” refers to the group heteroaryl-O—.

The term “heterocyclyl” refers to a monoradical saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclic substituent, such heterocyclic groups can be optionally substituted with 1, 2, 3, 4 or 5, and preferably 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR²⁰, where R²⁰ is alkyl, aryl, or heteroaryl and n is 0, 1 or 2.

The term “thiol” or “thio” refers to the group —SH.

The term “alkylthio” refers to the group —S-alkyl wherein alkyl is as defined herein.

The term “substituted alkylthio” refers to the group —S-substituted alkyl.

The term “arylthio” refers to the group —S-aryl, where aryl is as defined herein.

The term “heteroarylthiol” or “heteroarylthio” refers to the group —S-heteroaryl wherein the heteroaryl group is as defined above including optionally substituted heteroaryl groups as also defined above.

“Heterocyclylthio” refers to the group —S-heterocycyl.

The term “sulfoxide” refers to a group —S(O)R²⁶, in which R²⁶ is alkyl, aryl, or heteroaryl. “Substituted sulfoxide” refers to a group —S(O)R²⁷, in which R²⁷ is substituted alkyl, substituted aryl, or substituted heteroaryl, as defined herein.

The term “sulfone” refers to a group —S(O)₂R²⁸, in which R²⁸ is alkyl, aryl, or heteroaryl. “Substituted sulfone” refers to a group —S(O)₂R²⁹, in which R²⁹ is substituted alkyl, substituted aryl, or substituted heteroaryl, as defined herein.

The term “keto” or “oxo” refers to a group —C(O)—. The term “thiocarbonyl” refers to a group —C(S)—. The term “carboxy” refers to a group —C(O)—OH.

“Optional” or “optionally” means that the subsequently described event or circumstance may or may not occur, and that the description includes instances where said event or circumstance occurs and instances in which it does not.

The term “compound of Formula I, Formula II, or Formula III” is intended to encompass the compounds of the disclosure as disclosed, and the pharmaceutically acceptable salts, pharmaceutically acceptable esters, prodrugs, hydrates and polymorphs of such compounds. Additionally, the compounds of the disclosure may possess one or more asymmetric centers, and can be produced as a racemic mixture or as individual enantiomers or diastereoisomers. The number of stereoisomers present in any given compound of the disclosure depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis, or by resolution of the compound of the disclosure by conventional means. The individual stereoisomers (including individual enantiomers and diastereoisomers) as well as racemic and non-racemic mixtures of stereoisomers are encompassed within the scope of the present disclosure, all of which are intended to be depicted by the structures of this specification unless otherwise specifically indicated.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R—S system. When the compound is a pure enantiomer the stereochemistry at each chiral carbon may be specified by either R or S. Resolved compounds whose absolute configuration is unknown are designated (+) or (−) depending on the direction (dextro- or levorotary) which they rotate the plane of polarized light at the wavelength of the sodium D line.

The term “tautomer” refers to alternate forms of a compound that differ in the position of a proton, such as enol, keto, and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

The term “prodrug” as used herein, refers to compounds of Formula I, II or III that include chemical groups which, in vivo, can be converted and/or can be split off from the remainder of the molecule to provide for the active drug, a pharmaceutically acceptable salt thereof, or a biologically active metabolite thereof. Suitable groups are well known in the art and particularly include: for the carboxylic acid moiety, a prodrug selected from, e.g., esters including, but not limited to, those derived from alkyl alcohols, substituted alkyl alcohols, hydroxy substituted aryls and heteroaryls and the like; amides; hydroxymethyl, aldehyde and derivatives thereof. Structures of such prodrugs can be of Formula III shown below.

In many cases, the compounds of this disclosure are capable of forming acid and/or base salts by virtue of the presence of amino and/or carboxyl groups or groups similar thereto. The term “pharmaceutically acceptable salt” refers to salts that retain the biological effectiveness and properties of the compounds of Formula I, II, or III, and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### 2. NOMENCLATURE

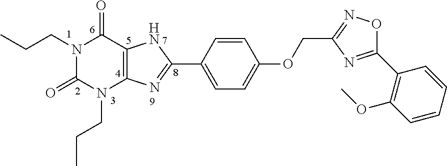

The naming and numbering of the compounds of the disclosure is illustrated with a representative compound of Formula I in which R¹ is n-propyl, R² is n-propyl, R³ is hydrogen, X is phenylene, Y is —O—(CH₂), and Z is 5-(2-methoxyphenyl)-[1,2,4]-oxadiazol-3-yl, which is named: 8-{4-[5-(2-methoxyphenyl)-[1,2,4]-oxadiazol-3-ylmethoxy]-phenyl}-1,3-dipropyl-1,3,7-trihydropurine-2,6-dione.

### 3. METHODS

As stated above, the present disclosure relates to methods of treating pulmonary hypertension. The method comprises administering to a patient in need thereof a therapeutically effective amount of an A_{2B} adenosine receptor antagonist.

### Pulmonary Hypertension, Classification and Clinical Parameters

The pulmonary hypertension condition treated by the methods of the disclosure can comprise any one or more of the conditions recognized according to the World Health Organization (WHO) or Dana Point, Calif. (2008) classification (see, for example, Simonneau et al., J Am Coll Cardio, 54(1):S43-54 (2009)):

1 Pulmonary arterial hypertension (PAH)
1.1. Idiopathic PAH
1.2. Heritable
1.2.1. Bone morphogenetic protein receptor type 2 (BMPR2)
1.2.2. Activin receptor-like kinase type 1 (ALK1), endoglin (with or without hereditary hemorrhagic telangiectasia)
1.2.3. Unknown
1.3. Drug- and toxin-induced
1.4. Associated with
1.4.1. Connective tissue diseases
1.4.2. Human immunodeficiency virus (HIV) infection
1.4.3. Portal Hypertension
1.4.4. Congenital heart diseases
1.4.5. Schistosomiasis
1.4.6. Chronic Hemolytic Anemia
1.5 Persistent pulmonary hypertension of the newborn

1′ Pulmonary veno-occlusive disease (PVOD) and/or pulmonary capillary hemangiomatosis (PCH)

2 Pulmonary hypertension owing to left heart disease
2.1. Systolic Dysfunction
2.2. Diastolic dysfunction
2.3. Valvular Disease

3 Pulmonary hypertension owing to lung diseases and/or hypoxia
3.1. Chronic obstructive pulmonary disease
3.2. Interstitial lung disease
3.3. Other pulmonary diseases with mixed restrictive and obstructive pattern
3.4. Sleep-disordered breathing
3.5. Alveolar hypoventilation disorders
3.6. Chronic exposure to high altitude
3.7. Developmental abnormalities

4. Chronic thromboembolic pulmonary hypertension (CTEPH)

5 Pulmonary hypertension with unclear multifactorial mechanisms
5.1. Hematologic disorders: myeloproliferative disorders, splenectomy
5.2. Systemic disorders: sarcoidosis, pulmonary Langerhans cell histiocytosis: lymphangioleiomyomatosis, neurofibromatosis, vasculitis
5.3. Metabolic disorders: glycogen storage disease, Gaucher disease, thyroid disorders
5.4. Others: tumoral obstruction, fibrosing mediastinitis, chronic renal failure on dialysis

In one aspect, the pulmonary hypertension condition comprises PAH (WHO Group 1), for example idiopathic PAH, familial PAH or PAH associated with another disease or condition.

Pulmonary hypertension at baseline can be mild, moderate or severe, as measured for example by WHO functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system:

Class I: pulmonary hypertension without resulting limitation of physical activity; ordinary physical activity does not cause undue dyspnea or fatigue, chest pain or near syncope;

Class II: pulmonary hypertension resulting in slight limitation of physical activity; patient comfortable at rest; ordinary physical activity causes undue dyspnea or fatigue, chest pain or near syncope;

Class III: pulmonary hypertension resulting in marked limitation of physical activity; patient comfortable at rest; less than ordinary activity causes undue dyspnea or fatigue, chest pain or near syncope;

Class IV: pulmonary hypertension resulting in inability to carry out any physical activity without symptoms; patient manifests signs of right-heart failure; dyspnea and/or fatigue may be present even at rest; discomfort is increased by any physical activity.

In one aspect, the methods are directed to treating Class I, also known as asymptomatic pulmonary hypertension.

In one aspect, the subject at baseline exhibits pulmonary hypertension (e.g., PAH) of at least WHO Class II, for example WHO Class II or Class III.

In another aspect, the subject at baseline exhibits mean PAP at rest of at least about 30 mmHg, for example at least about 35, at least about 40, at least about 45 or at least about 50 mmHg

The methods of the present disclosure, when applied to a subject, can achieve one or more of the following objectives:

(a) adjustment of one or more hemodynamic parameters towards a more normal level, for example lowering mean PAP or PVR, or raising Pulmonary Capillary Wedge Pressure (PCWP) or Left Ventricular End-Diastolic Pressure (LVEDP), versus baseline;

(b) improvement of pulmonary function versus baseline, for example increasing exercise capacity, illustratively as measured in a test of 6-minute walking distance (6MWD), or lowering Borg dyspnea index (BDI);

(c) improvement of one or more quality of life parameters versus baseline, for example an increase in score on at least one of the SF-36® health survey functional scales;

(d) general improvement versus baseline in the severity of the condition, for example by movement to a lower WHO functional class;

(e) improvement of clinical outcome following a period of treatment, versus expectation in absence of treatment (e.g., in a clinical trial setting, as measured by comparison with placebo), including improved prognosis, extending time to or lowering probability of clinical worsening, extending quality of life (e.g., delaying progression to a higher WHO functional class or slowing decline in one or more quality of life parameters such as SF-36® health survey parameters), and/or increasing longevity; and/or

(f) adjustment towards a more normal level of one or more molecular markers that can be predictive of clinical outcome (e.g., plasma concentrations of endothelin-1 (ET-1), cardiac troponin T (cTnT) or B-type natriuretic peptide (BNP)).

What constitutes a therapeutically effective amount of A_{2B} adenosine receptor antagonist for treating pulmonary hypertension, or in particular, PAH, can vary depending on the particular pulmonary hypertension condition to be treated, the severity of the condition, body weight and other parameters of the individual subject, and can be readily established without undue experimentation by the physician or clinician based on the disclosure herein. However, contemplated doses are described below.

Various clinical parameters and standards to measure the effectiveness of a pulmonary hypertension therapy are described below and are known in the art as well. Accordingly, the effectiveness of A_{2B} adenosine receptor antagonist can be measured by these parameters or standards. Additionally, the relative effectiveness of A_{2B} adenosine receptor antagonist, as compared to other agents, can be determined with these clinical parameters or standards, as well as in a non-clinical setting. Examples of such non-clinical settings include, without limitation, an animal model. Non-limiting examples of animal models are provided in Examples.

### A. Improvement on Clinical Parameters

In one aspect, the subject being treated experiences, during or following the treatment period, at least one of

(a) adjustment of one or more hemodynamic parameters indicative of the pulmonary hypertension condition towards a more normal level versus baseline;

(b) increase in exercise capacity versus baseline;

(c) lowering of Borg Dyspnea Index (BDI) versus baseline;

(d) improvement of one or more quality of life parameters versus baseline; and/or

(e) movement to a lower WHO functional class.

Any suitable measure of exercise capacity can be used; a particularly suitable measure is obtained in a 6-minute walk test (6MWT), which measures how far the subject can walk in 6 minutes, i.e., the 6-minute walk distance (6MWD).

The Borg dyspnea index (BDI) is a numerical scale for assessing perceived dyspnea (breathing discomfort). It measures the degree of breathlessness after completion of the 6 minute walk test (6MWT), where a BDI of 0 indicates no breathlessness and 10 indicates maximum breathlessness.

In various aspects, an effective amount of a pulmonary hypertension therapy adjusts one or more hemodynamic parameters indicative of the pulmonary hypertension condition towards a more normal level. In one such aspect, mean PAP is lowered, for example by at least about 3 mmHg, or at least about 5 mmHg, versus baseline. In another such aspect, PVR is lowered. In yet another such aspect, PCWP or LVEDP is raised.

In various aspects, an effective amount of a pulmonary hypertension therapy improves pulmonary function versus baseline. Any measure of pulmonary function can be used; illustratively 6MWD is increased or BDI is lowered.

In one such aspect, 6MWD is increased from baseline by at least about 10 meters, for example at least about 20 meters or at least about 30 meters. In many instances, the method of the present embodiment will be found effective to increase 6MWD by as much as 50 meters or even more.

In another such aspect, BDI, illustratively as measured following a 6MWT, is lowered from baseline by at least about 0.5 index points. In many instances, the method of the present embodiment will be found effective to lower BDI by as much as 1 full index point or even more.

The SF-36® health survey provides a self-reporting, multi-item scale measuring eight health parameters: physical functioning, role limitations due to physical health problems, bodily pain, general health, vitality (energy and fatigue), social functioning, role limitations due to emotional problems, and mental health (psychological distress and psychological well-being). The survey also provides a physical component summary and a mental component summary.

In various aspects, an effective amount of a pulmonary hypertension therapy can improve quality of life of the subject, illustratively as measured by one or more of the health parameters recorded in an SF-36® survey. For example, an improvement versus baseline is obtained in at least one of the SF-36® physical health related parameters (physical health, role-physical, bodily pain and/or general health) and/or in at least one of the SF-36® mental health related parameters (vitality, social functioning, role-emotional and/or mental health). Such an improvement can take the form of an increase of at least 1, for example at least 2 or at least 3 points, on the scale for any one or more parameters.

### B. Improvement of Prognosis

In another embodiment, the treatment method of the present disclosure can improve the prognosis for a subject having a pulmonary hypertension condition. The treatment of this embodiment can provide (a) a reduction in probability of a clinical worsening event during the treatment period, and/or (b) a reduction from baseline in serum brain natriuretic peptide (BNP) concentration, wherein, at baseline, time from first diagnosis of the condition in the subject is not greater than about 2 years.

Time from first diagnosis, in various aspects, can be, for example, not greater than about 1.5 years, not greater than about 1 year, not greater than about 0.75 year or not greater than about 0.5 year. In one aspect, administration of A_{2B} adenosine receptor antagonist can begin substantially immediately, for example, within about one month or within about one week, upon diagnosis.

In this embodiment, the treatment period is long enough for the stated effect to be produced. Typically, the longer the treatment continues, the greater and more lasting will be the benefits. Illustratively, the treatment period can be at least about one month, for example at least about 3 months, at least about 6 months or at least about 1 year. In some cases, administration can continue for substantially the remainder of the life of the subject.

Clinical worsening event (CWEs) include death, lung transplantation, hospitalization for the pulmonary hypertension condition, atrial septostomy, initiation of additional pulmonary hypertension therapy or an aggregate thereof. Therefore, the treatments of the present disclosure can be effective to provide a reduction of at least about 25%, for example at least about 50%, at least about 75% or at least about 80%, in probability of death, lung transplantation, hospitalization for pulmonary arterial hypertension, atrial septostomy and/or initiation of additional pulmonary hypertension therapy during the treatment period.

Time to clinical worsening of the pulmonary hypertension condition is defined as the time from initiation of an A_{2B} adenosine receptor antagonist treatment regime to the first occurrence of a CWE.

The pulmonary hypertension condition according to this embodiment can comprise any one or more of the conditions in the WHO or Venice (2003) classification described above. In one aspect, the condition comprises PAH (WHO Group 1), for example idiopathic PAH, familial PAH or PAH associated with another disease.

In various aspects of this embodiment, the subject at baseline exhibits PH (e.g., PAH) of at least WHO Class II, for example Class II, Class III or Class IV as described above.

In a more particular embodiment, the subject at baseline has a resting PAP of at least about 30 mmHg, for example at least about 35 mmHg or at least about 40 mmHg

### C. Prolongation of Life

In yet another embodiment, the treatment methods of the present disclosure can prolong the life of a subject having a pulmonary hypertension condition, from a time of initiation of treatment, by at least about 30 days. Variants and illustrative modalities of this method are as set forth above.

### D. Extending Time to Clinical Worsening

Still in another embodiment, the present methods can extend time to clinical worsening in a subject having a pulmonary hypertension condition, and decrease the probability of a clinical worsening event by at least about 25%. Variants and illustrative modalities of this method are as set forth above.

### E. Other Treatment Objectives

In any of the methods described hereinabove, the subject can be male or female. For example, the A_{2B} adenosine receptor antagonist can be administered to a female subject according to any of the above methods, including the indicated variants and illustrative modalities thereof. Alternatively, the A_{2B} adenosine receptor antagonist can be administered to a male subject, for example a reproductively active male subject, according to any of the above methods, including the indicated variants and illustrative modalities thereof.

In another embodiment, the methods provided herein are useful for treating a pulmonary hypertension condition in a reproductively active male subject, wherein fertility of the subject is not substantially compromised. “Not substantially compromised” in the present context means that spermatogenesis is not substantially reduced by the treatment and that no hormonal changes are induced that are indicative of or associated with reduced spermatogenesis. Male fertility can be assessed directly, for example, by sperm counts from semen samples, or indirectly by changes in hormones such as follicle stimulating hormone (FSH), luteinizing hormone (LH), inhibin B and testosterone.

In one embodiment, a method is provided for treating PAH in a subject, wherein the PAH is associated with one or more of (a) a congenital heart defect, (b) portal hypertension, (c) use of a drug or toxin other than an anorexigen, (d) thyroid disorder, (e) glycogen storage disease, (f) Gaucher disease, (g) hereditary hemorrhagic telangiectasia, (h) hemoglobinopathy, (i) myeloproliferative disorder, (j) splenectomy, (k) pulmonary veno-occlusive disease and/or (l) pulmonary capillary hemangiomatosis. Variants and illustrative modalities of this method are as set forth hereinabove.

Further, in another embodiment, a method is provided for treating a pulmonary hypertension condition classified in WHO Groups 2-5 in a subject. In a particular embodiment, the pulmonary hypetension condition is classified in WHO Group 3. Variants and illustrative modalities of this method are as set forth hereinabove. In one aspect, the condition comprises left-sided atrial or ventricular heart disease and/or left-sided valvular heart disease. In another aspect, the condition is associated with one or more of chronic obstructive pulmonary disease (COPD), interstitial lung disease (ILD), sleep-disordered breathing, an alveolar hypoventilation disorder, chronic exposure to high altitude, a developmental abnormality, thromboembolic obstruction of proximal and/or distal pulmonary arteries, a non-thrombotic pulmonary embolism, sarcoidosis, histiocytosis X, lymphangiomatosis, and/or compression of pulmonary vessels.

As discussed below, A_{2B} adenosine receptor antagonist can be administered in a variety of manners.

### Methods of Treating Pulmonary Hypertension

Several factors have been implicated in the pathogenesis of pulmonary hypertension including: 1) vascular remodeling, such as intimate wall thickening; 2) hyperproliferation in human pulmonary arterial smooth muscle cells (HPASM) and human pulmonary endothelial cells (HPAEC); 3) elevated levels of cytokines, including inflammatory cytokines IL-6 (Steiner, et al. (2009)), IL-8, endothelin, thromboxame in HPASM and HPAEC.

Group 3 of PH is often associated with underlying chronic lung diseases such as chronic obstructive pulmonary disease (COPD) and pulmonary fibrosis. This group includes chronic bronchiectasis, cystic fibrosis, and a newly identified syndrome characterized by the combination of pulmonary fibrosis, mainly of the lower zones of the lung, and emphysema, mainly of the upper zones of the lung.

It has now been found that several of the factors associated with pulmonary hypertension may be treated by administration of A_{2B} adenosine receptor antagonist. In particular, it has been discovered that vascular remodeling in the form of wall thickening, and proliferation in pulmonary tissue may be attenuated by administration of an A_{2B} adenosine receptor antagonist. Further, it has been discovered that administration of an A_{2B} adenosine receptor antagonist to either HPASMs and HPAECs reduces the level of IL-6, additional inflammatory molecules, such as granular colony-stimulating factor (G-CSF), and/or chemokines, such as IL-8. Still further, it has been discovered the proliferation and migration of HPASM is inhibited by administration of an A_{2B} adenosine receptor antagonist.

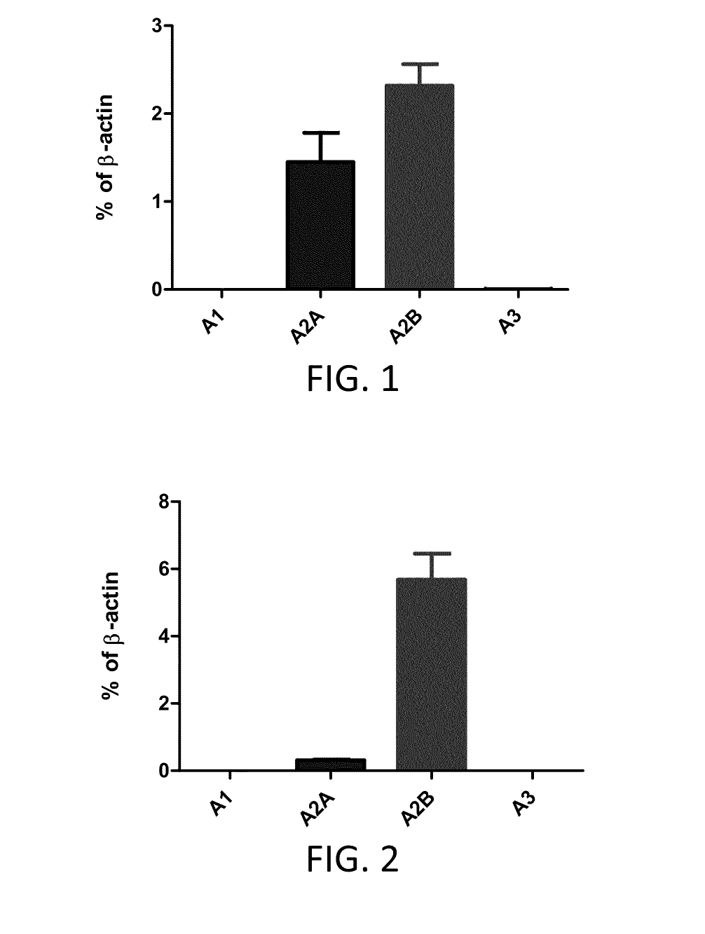

These findings are premised on the surprising and unexpected discovery that the A_{2B} adenosine receptors are highly expressed in both HPASM and HPAEC. The results of this are presented in FIG. 1 and FIG. 2 obtained by the protocol in Example 3. In fact, the A_{2B} receptor subtype is expressed much more highly than the other three subtypes, including A₁, A_{2A}, and A₃. To substantiate that pulmonary hypertension could be treated with an A_{2B} adenosine receptor antagonist, several in vivo tests were conducted.

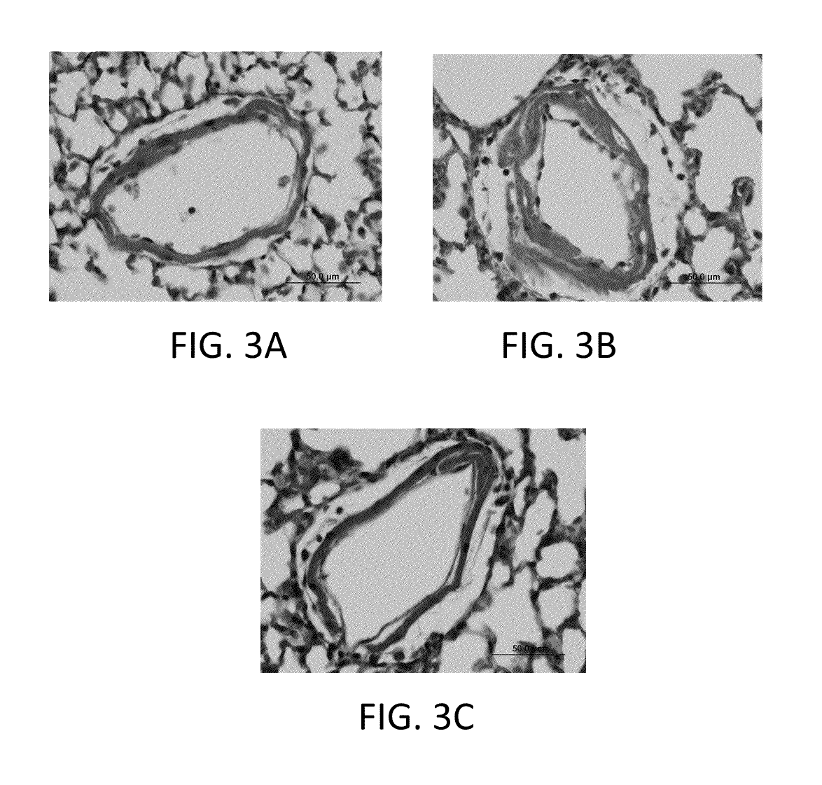

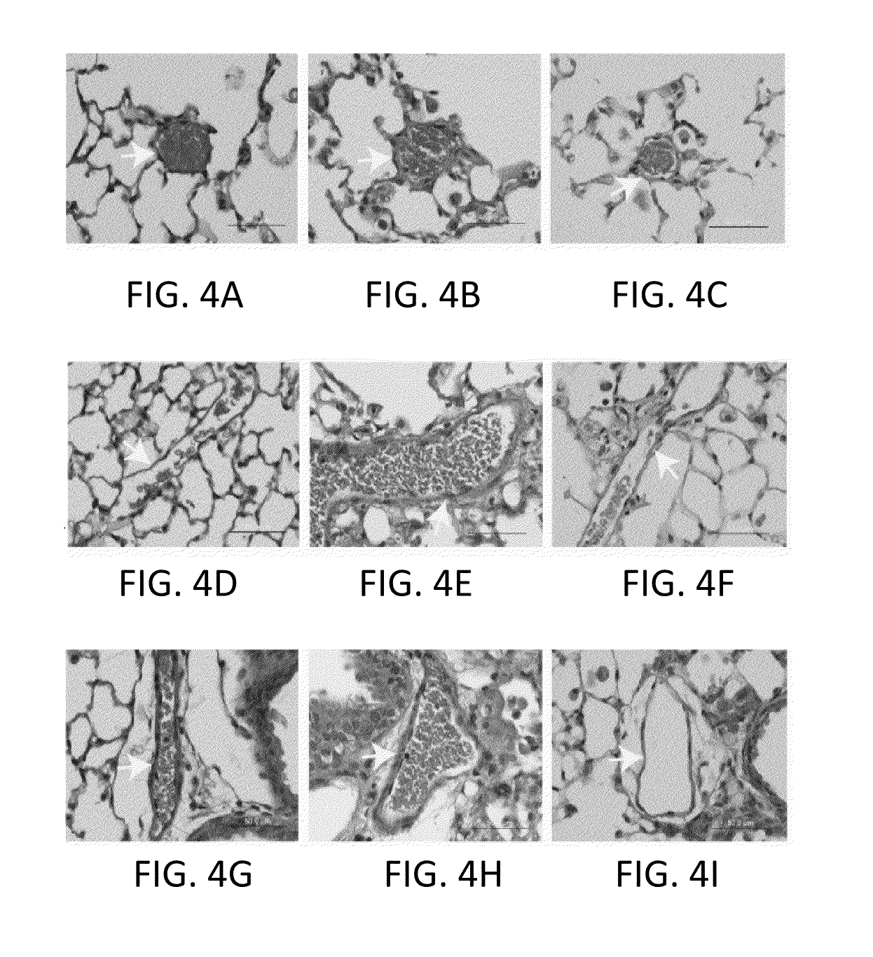

First, animal models were examined to determine if vascular wall thickening could be attenuated with treatment of an A_{2B} antagonist. As can be seen in FIGS. 3 and 4 and as described in Examples 4 and 13, Compound A attenuated the vascular wall thickening in the ADA knock-out mouse and the A_{2B receptor KO mice exposed to bleomycin no longer develop the vascular wall thickening suggesting that the A}_{2B} receptor is critical in the pathogenesis of pulmonary hypertension.

Second, HPAECs and HPASMs were examined to determine whether activation of the A_{2B} receptor followed by deactivation of that receptor with an A_{2B} antagonist would affect the release of various cytokines and chemokines associated with inflammation, and other proteins associated with remodeling and proliferation. In these examples, cells were treated with N-ethylcarboxamide adenosine (NECA), which is a stable A₁ and A₂ receptor agonist. The protein activity was measured after administration NECA and then again after administration of the A_{2B} receptor antagonist.

It was surprisingly found that ET-1, a potent vasoconstrictor, was dose-dependently increased by the adenosine agonist and then was significantly reduced by administration of Compound A. See, Example 6, FIG. 6. Similarly, it was found that thromboxane B2 release in HPASM was reduced by Compound A. See, Example 8, FIG. 11. These findings suggest that activation of the A_{2B} receptor induces the release of ET-1 and thromboxane B2. Therefore, by inhibiting the release of ET-1 and thromboxane, it is contemplated that potential vascular remodeling due to vasoconstriction may also be inhibited.

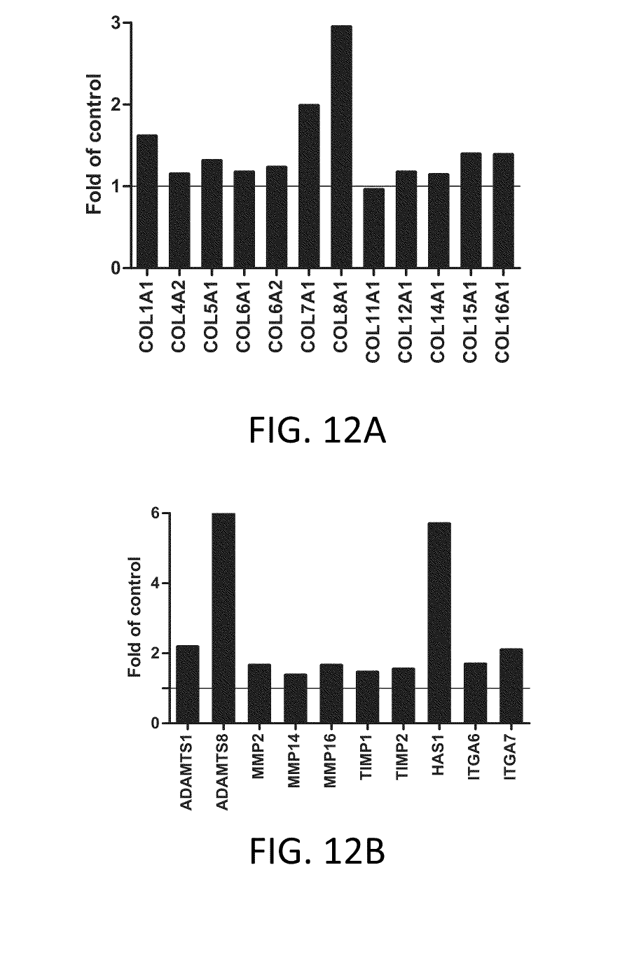

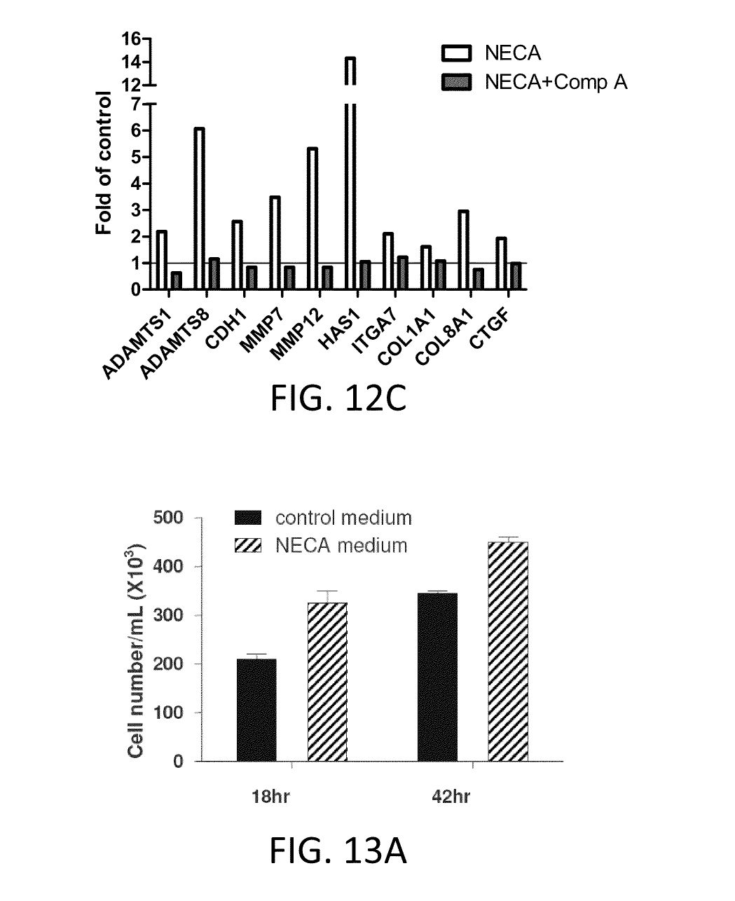

As it relates to vascular remodeling, it has also been found that expression of certain collagen, extracellular matrix proteins, and extracellular matrix enzymes (e.g., ADAMTS1, ADAMTS8, CDH1, MMPI, MMP12, HAS1, ITGA7, COL1A1, COL8A1 and CTGF) was decreased by administration of Compound A (FIG. 12A-C). This suggests that activation of the A_{2B} receptor induces release of those genes associated with tissue remodeling.

Reduction in the release of IL-8, a chemokine that is a major mediator in the inflammatory response, was seen in both HPAEC and HPASM. It is contemplated that by reduction IL-8, a proposed component of the inflammatory mechanism of pulmonary hypertension can also be inhibited.

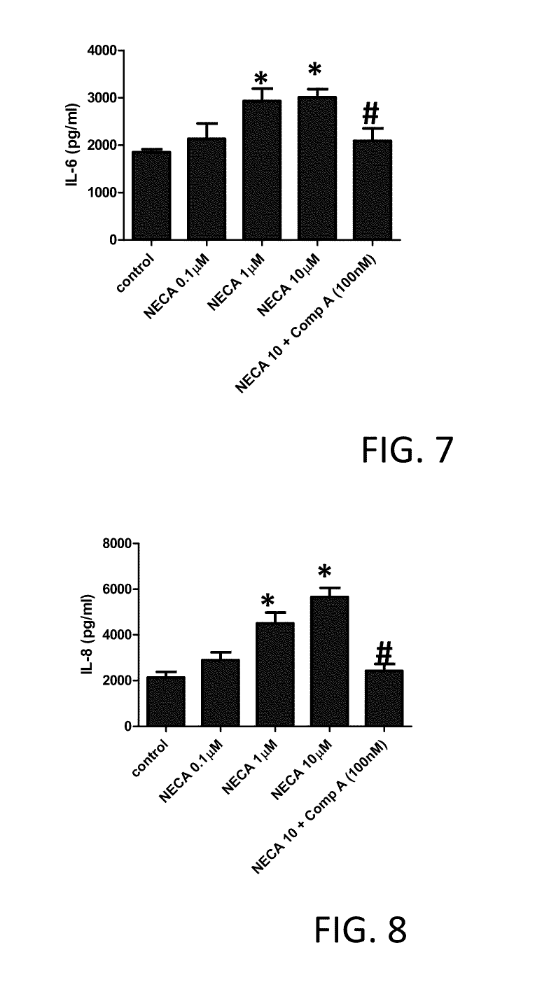

Reduction of the release of inflammatory cytokines, IL-6 and G-CSF (granular colony stimulating factor) was observed after administration of Compound A (FIG. 7-9). These findings suggest that the activation of the A_{2B} receptor induces the release of these cytokines. This further suggests that the inflammatory component of pulmonary hypertension may be modulated by the antagonists described herein.

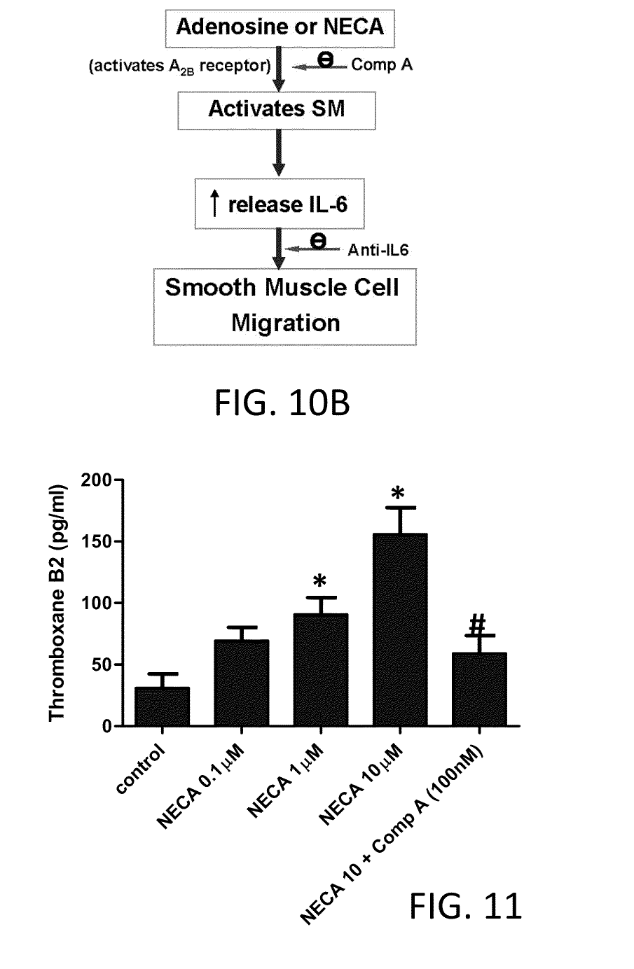

It has also been observed that through activating A_{2B} adenosine receptor, NECA activates smooth muscle which releases IL-6 which in turn enhances smooth muscle cell migration (FIG. 10A-B). Such enhancement, as observed, was inhibited by Compound A (FIG. 10A).

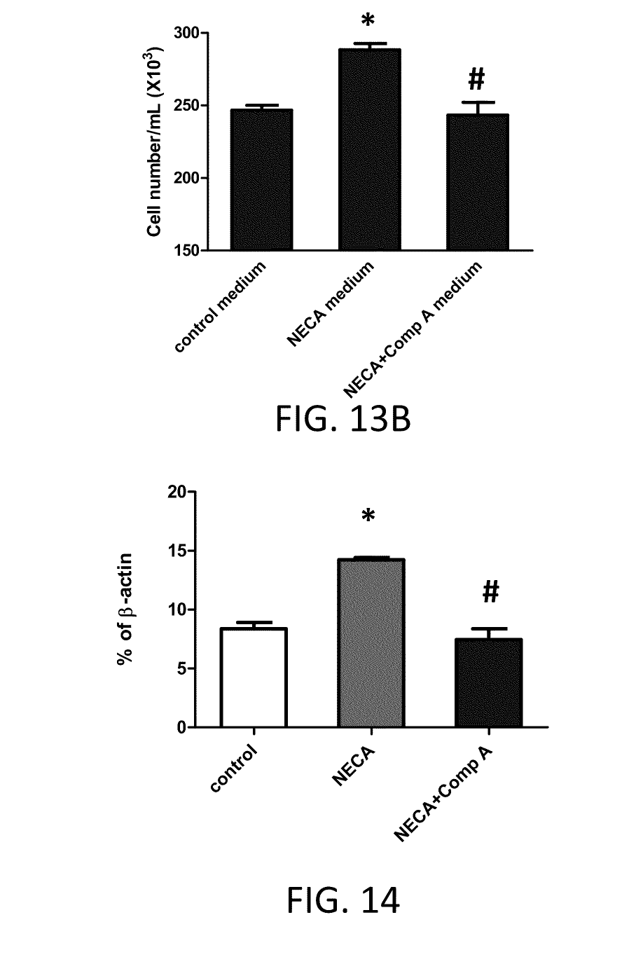

As it relates to proliferation of smooth muscle cells, it was observed that both Compound A and ambrisentan, a known antagonist of an endothelin receptor, reduced the proliferation after induction by the agonist (FIG. 13A-B). As noted above, Compound A inhibited the release of ET-1. Therefore, when treated either with Compound A alone or in combination with a known endothelin antagonists, proliferation may be reduced (FIG. 13C).

To further substantiate that A_{2B} adenosine receptor antagonists treat pulmonary hypertension, smooth muscles cells were tested for expression of NOTCH3. It is contemplated that pulmonary hypertension is characterized by an overexpression of NOTCH3 in small pulmonary artery smooth muscle cells. Further, the severity of the disease may also be correlated with the amount of NOTCH3 protein in the lung. See, Li, X., et al., “Notch3 signaling promotes the development of pulmonary arterial hypertension” Nature Medicine, 15(11):1289-1297 (2009). As can be seen in FIG. 14, agonists induced expression of NOTCH3 was reduced by administration of the antagonist in smooth muscle cells.

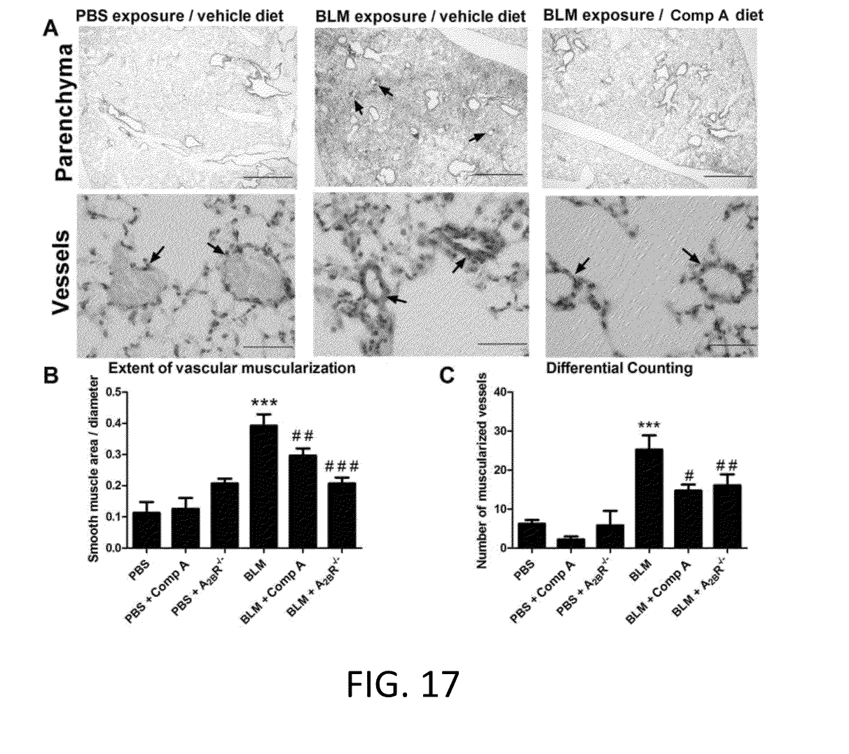

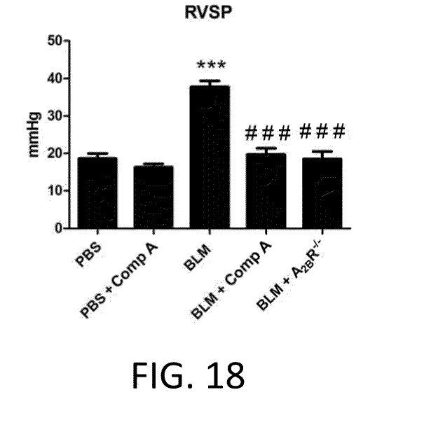

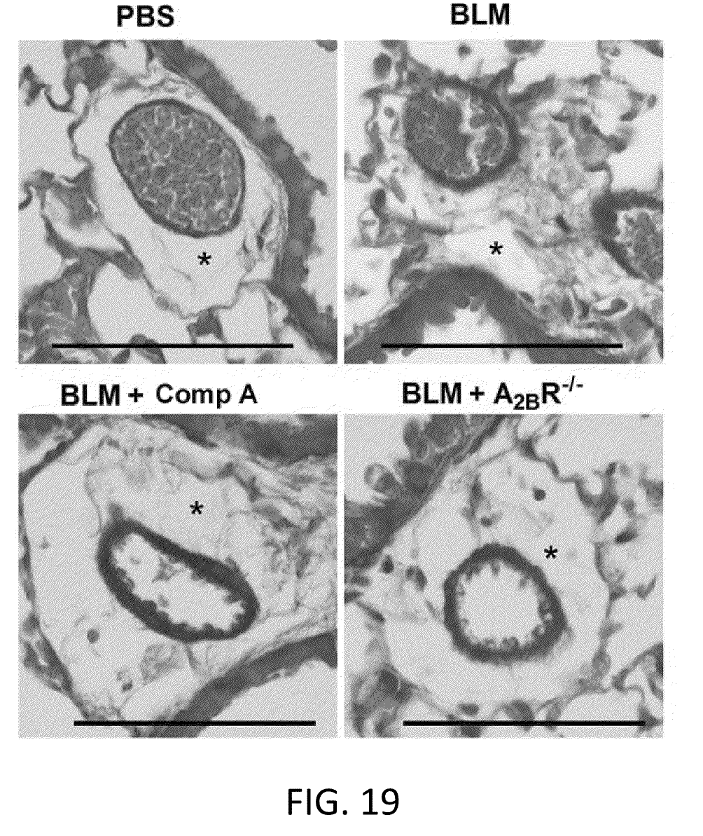

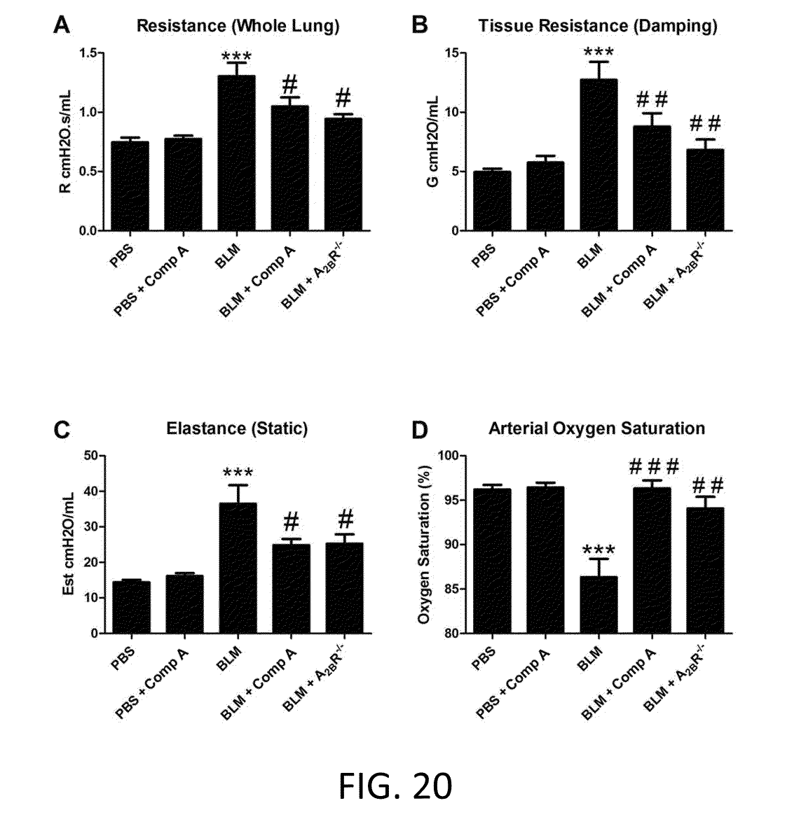

In a preclinical model of pulmonary hypertension owing to lung diseases (Group 3 of PH), Compound A has been shown to reduce vasculopathy and right ventricular systolic pressure (RVSP) (FIG. 18), to improve pulmonary vascular remodeling (FIG. 17), to inhibit fibrosis (FIG. 19), and to reduce the release of cytokines and ET-1 and improve lung functions (FIG. 20-22). Therefore, these results highlight the role of the A_{2B} receptor in the pathogenesis of pulmonary hypertension associated with chronic lung injury and confirm the A_{2B} receptor antagonists for the treatment of pulmonary hypertension.

Thus, it is now contemplated that pulmonary hypertension, in particular PAH and Group 3 of pulmonary hypertension, both the underlying disease and the inflammatory component, may be treated by administration of an A_{2B} adenosine receptor antagonist. Therefore, in one embodiment is provided a method of treating pulmonary hypertension in a patient in need thereof, said method comprising administering to the patient a therapeutically effective amount of an A_{2B} adenosine receptor antagonist.

In one embodiment of the disclosure the pulmonary arterial hypertension is selected from idiopathic PAH, familial PAH, or PAH associated with another disease or condition. In another embodiment, the method is for the treatment of pulmonary inflammation. In one embodiment, the patient is human.

As more thoroughly described below, the antagonists may be administered in a variety of ways, including, systemic, oral, intravenous, intramuscular, intraperitoneal, and inhalation.

### 4. A_{2B} ADENOSINE RECEPTOR ANTAGONISTS

In one aspect, the disclosure provides methods for treating pulmonary hypertension by administering an A_{2B} adenosine receptor antagonist to the patient in need thereof. An A_{2B} adenosine receptor antagonist is any compound that inhibits or otherwise modulates the activity of the A_{2B} receptor. A_{2B} adenosine receptor antagonists are known in the art. For example, several small molecule inhibitors of the receptor have been identified. Exemplary compounds include:

| Compound Structure | Chemical Name | Source |
|---|---|---|
| 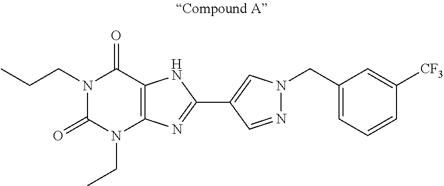 | 3-ethyl-1-propyl- 8-(1-(3- (trifluoromethyl) benzyl)-1H- pyrazol-4-yl)-1H- purine- 2,6(3H,7H)-dione | U.S. Pat. No. 6,825,349 |
| 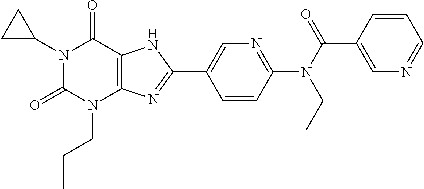 | N-[5-(1- cyclopropyl-2,6- dioxo-3-propyl- 2,3,6,7- tetrahydro-1H- purin-8-yl)- pyridin-2-yl]-N- ethyl- nicotinamide | US Published Patent Application 2007/0072843 |
| 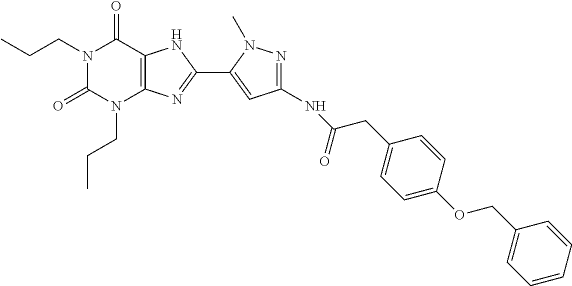 | 2-(4- (benzyloxy)phen- yl)-N-(5-(2,6- dioxo-1,3- dipropyl-2,3,6,7- tetrahydro-1H- purin-8-yl)-1- methyl-1H- pyrazol-3- yl)acetamide | US Published Patent Application 2007/0072843 |

Additional A_{2B} adenosine receptor antagonists are 8-cyclic xanthine derivative, where the cyclic substituent may be aryl, heteroaryl, cycloalkyl, or heterocyclic all of which cyclic groups are optionally substituted as defined above. Examples of 8-cyclic xanthine derivatives may be found throughout the literature, see, e.g., Baraldi, P. et al. “Design, Synthesis, and Biological Evaluation of New 8-Heterocyclic Xanthine Derivatives as Highly Potent and Selective Human A_{2B} adenosine receptor antagonists”, J. Med. Chem., (2003), also found in WO02/42298, WO03/02566, WO2007/039297, WO02/42298, WO99/42093, WO2009/118759, and WO2006/044610 which are all incorporated by reference in their entirety.

A variety of A_{2B} adenosine receptor antagonists are contemplated to be useful in this disclosure. The compounds are described in U.S. Pat. Nos. 6,825,349, 7,105,665, and 6,997,300, which are all incorporated by reference in their entirety. In one embodiment, the disclosure is directed to use of a compound of Formula I or II.

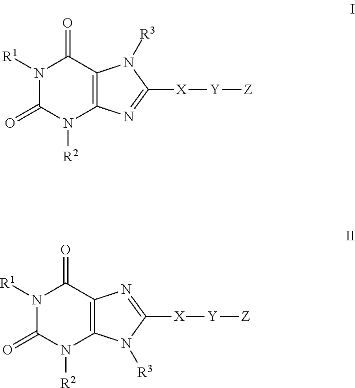

wherein:
R¹ and R² are independently chosen from hydrogen, optionally substituted alkyl, or a group -D-E, in which D is a covalent bond or alkylene, and E is optionally substituted alkoxy, optionally substituted cycloalkyl, optionally substituted aryl, optionally substituted heteroaryl, optionally substituted heterocyclyl, optionally substituted alkenyl or optionally substituted alkynyl, with the proviso that when D is a covalent bond E cannot be alkoxy;
R³ is hydrogen, optionally substituted alkyl or optionally substituted cycloalkyl;
X is optionally substituted arylene or optionally substituted heteroarylene;
Y is a covalent bond or alkylene in which one carbon atom can be optionally replaced by —O—, —S—, or —NH—, and is optionally substituted by hydroxy, alkoxy, optionally substituted amino, or —COR¹⁶, in which R¹⁶ is hydroxy, alkoxy or amino;
   with the proviso that when the optional substitution is hydroxy or amino it cannot be adjacent to a heteroatom; and
Z is optionally substituted monocyclic aryl or optionally substituted monocyclic heteroaryl; or
Z is hydrogen when X is optionally substituted heteroarylene and Y is a covalent bond;
   with the proviso that when X is optionally substituted arylene, Z is optionally substituted monocyclic heteroaryl
   or a pharmaceutically acceptable salt, tautomer, isomer, a mixture of isomers, or prodrug thereof.

In one embodiment, compounds of Formula I and II are those in which R¹ and R² are independently hydrogen, optionally substituted lower alkyl, or a group -D-E, in which D is a covalent bond or alkylene, and E is optionally substituted phenyl, optionally substituted cycloalkyl, optionally substituted alkenyl, or optionally substituted alkynyl, particularly those in which R³ is hydrogen.

Within this group, a first class of compounds include those in which R¹ and R² are independently lower alkyl optionally substituted by cycloalkyl, preferably n-propyl, and X is optionally substituted phenylene. Within this class, a subclass of compounds are those in which Y is alkylene, including alkylene in which a carbon atom is replaced by oxygen, preferably —O—CH₂—, more especially where the oxygen is the point of attachment to phenylene. Within this subclass, in one embodiment, Z is optionally substituted oxadiazole, particularly optionally substituted [1,2,4]-oxadiazol-3-yl, especially [1,2,4]-oxadiazol-3-yl substituted by optionally substituted phenyl or by optionally substituted pyridyl.

A second class of compounds include those in which X is optionally substituted 1,4-pyrazolene. Within this class, a subclass of compounds are those in which Y is a covalent bond, alkylene, lower alkylene, and Z is hydrogen, optionally substituted phenyl, optionally substituted pyridyl or optionally substituted oxadiazole. Within this subclass, one embodiment includes compounds in which R¹ is lower alkyl optionally substituted by cycloalkyl, and R² is hydrogen. Another embodiment includes those compounds in which Y is —(CH₂)— or —CH(CH₃)— and Z is optionally substituted phenyl, or Y is —(CH₂)— or —CH(CH₃)— and Z is optionally substituted oxadiazole, particularly 3,5-[1,2,4]-oxadiazole, or Y is —(CH₂)— or —CH(CH₃)— and Z is optionally substituted pyridyl. Within this subclass, also included are those compounds in which R¹ and R² are independently lower alkyl optionally substituted by cycloalkyl, especially n-propyl. In other embodiments are those compounds in which Y is a covalent bond, —(CH₂)— or —CH(CH₃)— and Z is hydrogen, optionally substituted phenyl, or optionally substituted pyridyl, particularly where Y is a covalent bond and Z is hydrogen.

At present, the compounds useful in this disclosure include, but are not limited to:
1-propyl-8-(1-{[3-(trifluoromethyl)phenyl]-methyl}pyrazol-4-yl)-1,3,7-trihydropurine-2,6-dione;
1-propyl-8-[1-benzylpyrazol-4-yl]-1,3,7-trihydropurine-2,6-dione;
1-butyl-8-(1-{[3-fluorophenyl]methyl}pyrazol-4-yl)-1,3,7-trihydropurine-2,6-dione;
1-propyl-8-[1-(phenylethyl)pyrazol-4-yl]-1,3,7-trihydropurine-2,6-dione;
8-(1-{[5-(4-chlorophenyl)(1,2,4-oxadiazol-3-yl)]methyl}pyrazol-4-yl)-1-propyl-1,3,7-trihydropurine-2,6-dione;
8-(1-{[5-(4-chlorophenyl)(1,2,4-oxadiazol-3-yl)]methyl}pyrazol-4-yl)-1-butyl-1,3,7-trihydropurine-2,6-dione;
1,3-dipropyl-8-pyrazol-4-yl-1,3,7-trihydropurine-2,6-dione;
1-methyl-3-sec-butyl-8-pyrazol-4-yl-1,3,7-trihydropurine-2,6-dione;
1-cyclopropylmethyl-3-methyl-8-{1-[(3-trifluoromethylphenyl)methyl]pyrazol-4-yl}-1,3,7-trihydropurine-2,6-dione;
1,3-dimethyl-8-{1-[(3-fluorophenyl)methyl]pyrazol-4-yl}-1,3,7-trihydropurine-2,6-dione;
3-methyl-1-propyl-8-{1-[(3-trifluoromethylphenyl)methyl]pyrazol-4-yl}-1,3,7-trihydropurine-2,6-dione;
3-ethyl-1-propyl-8-{1-[(3-trifluoromethylphenyl)methyl]pyrazol-4-yl}-1,3,7-trihydropurine-2,6-dione;
1,3-dipropyl-8-(1-{[3-(trifluoromethyl)phenyl]methyl}pyrazol-4-yl)-1,3,7-trihydropurine-2,6-dione;
1,3-dipropyl-8-{1-[(3-fluorophenyl)methyl]pyrazol-4-yl}-1,3,7-trihydropurine-2,6-dione;
1-ethyl-3-methyl-8-{1-[(3-fluorophenyl)methyl]pyrazol-4-yl}-1,3,7-trihydropurine-2,6-dione;
1,3-dipropyl-8-{1-[(2-methoxyphenyl)methyl]pyrazol-4-yl}-1,3,7-trihydropurine-2,6-dione;
1,3-dipropyl-8-(1-{[3-(trifluoromethyl)-phenyl]ethyl}pyrazol-4-yl)-1,3,7-trihydropurine-2,6-dione;
1,3-dipropyl-8-{1-[(4-carboxyphenyl)methyl]pyrazol-4-yl}-1,3,7-trihydropurine-2,6-dione;
2-[4-(2,6-dioxo-1,3-dipropyl(1,3,7-trihydropurin-8-yl))pyrazolyl]-2-phenylacetic acid;
8-{4-[5-(2-methoxyphenyl)-[1,2,4]oxadiazol-3-ylmethoxy]phenyl}-1,3-dipropyl-1,3,7-trihydropurine-2,6-dione;
8-{4-[5-(3-methoxyphenyl)-[1,2,4]oxadiazol-3-ylmethoxy]phenyl}-1,3-dipropyl-1,3,7-trihydropurine-2,6-dione;
8-{4-[5-(4-fluorophenyl)-[1,2,4]oxadiazol-3-ylmethoxy]phenyl}-1,3-dipropyl-1,3,7-trihydropurine-2,6-dione:
1-(cyclopropylmethyl)-8-[1-(2-pyridylmethyl)pyrazol-4-yl]-1,3,7-trihydropurine-2,6-dione;
1-n-butyl-8-[1-(6-trifluoromethylpyridin-3-ylmethyl)pyrazol-4-yl]-1,3,7-trihydropurine-2,6-dione;
8-(1-{[3-(4-chlorophenyl)(1,2,4-oxadiazol-5-yl)]methyl}pyrazol-4-yl)-1,3-dipropyl-1,3,7-trihydropurine-2,6-dione;
1,3-dipropyl-8-[1-({5-[4-(trifluoromethyl)phenyl]isoxazol-3-yl}methyl)pyrazol-4-yl]-1,3,7-trihydropurine-2,6-dione;
1,3-dipropyl-8-[1-(2-pyridylmethyl)pyrazol-4-yl]-1,3,7-trihydropurine-2,6-dione;
3-{[4-(2,6-dioxo-1,3-dipropyl-1,3,7-trihydropurin-8-yl)pyrazolyl]methyl}benzoic acid;
1,3-dipropyl-8-(1-{[6-(trifluoromethyl)(3-pyridyl)]methyl}pyrazol-4-yl)-1,3,7-trihydropurine-2,6-dione;
1,3-dipropyl-8-{1-[(3-(1H-1,2,3,4-tetraazol-5-yl)phenyl)methyl]pyrazol-4-yl}-1,3,7-trihydropurine-2,6-dione;
6-{[4-(2,6-dioxo-1,3-dipropyl-1,3,7-trihydropurin-8-yl)pyrazolyl]methyl}pyridine-2-carboxylic acid;
3-ethyl-1-propyl-8-[1-(2-pyridylmethyl)pyrazol-4-yl]-1,3,7-trihydropurine-2,6-dione;
8-(1-{[5-(4-chlorophenyl)isoxazol-3-yl]methyl}pyrazol-4-yl)-3-ethyl-1-propyl-1,3,7-trihydropurine-2,6-dione;
8-(1-{[3-(4-chlorophenyl)(1,2,4-oxadiazol-5-yl)]methyl}pyrazol-4-yl)-3-ethyl-1-propyl-1,3,7-trihydropurine-2,6-dione;
3-ethyl-1-propyl-8-(1-{[6-(trifluoromethyl)(3-pyridyl)]methyl}pyrazol-4-yl)-1,3,7-trihydropurine-2,6-dione;
1-(cyclopropylmethyl)-3-ethyl-8-(1-{[6-(trifluoromethyl)(3-pyridyl)]methyl}pyrazol-4-yl)-1,3,7-trihydropurine-2,6-dione; and
3-ethyl-1-(2-methylpropyl)-8-(1-{[6-(trifluoromethyl)(3-pyridyl)]methyl}pyrazol-4-yl)-1,3,7-trihydropurine-2,6-dione
or a pharmaceutically acceptable salt, tautomer, isomer, a mixture of isomers, or prodrug thereof.

It is contemplated that prodrugs of the above-described A_{2B} adenosine receptor antagonists are also useful in the methods of the disclosure. Exemplary prodrugs are taught in U.S. Pat. No. 7,625,881, which is hereby incorporated by reference in its entirety. Therefore, in one embodiment, the compounds useful in the methods of the disclosure include prodrugs of Formula III having the formula:

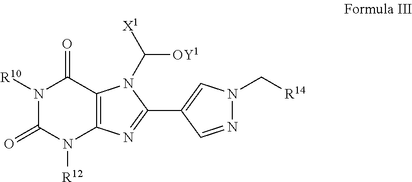

wherein:
R¹⁰ and R¹² are independently lower alkyl;
R¹⁴ is optionally substituted phenyl;
X¹ is hydrogen or methyl; and
Y¹ is —C(O)R¹⁷, in which R¹⁷ is independently optionally substituted lower alkyl, optionally substituted aryl, or optionally substituted heteroaryl; or
Y¹ is —P(O)(OR¹⁵)₂, in which R¹⁵ is hydrogen or lower alkyl optionally substituted by phenyl or heteroaryl;
   and the pharmaceutically acceptable salts thereof.

One group of compounds of Formula III are those in which R¹⁰ and R¹² are ethyl or n-propyl, especially those compounds in which R¹⁰ is n-propyl and R¹² is ethyl. In another embodiment, R¹⁴ is 3-(trifluoromethyl)phenyl and X¹ is hydrogen.

One subgroup includes those compounds of Formula III in which Y¹ is —C(O)R¹⁷, particularly those compounds in which R¹⁷ is methyl, ethyl, n-propyl, isopropyl, n-butyl, isobutyl, t-butyl, or n-pentyl, more particularly where R¹⁷ is methyl, n-propyl, or t-butyl. Another subgroup includes those compounds of Formula III in which Y¹ is —P(O)(OR¹⁵)₂, especially where R¹⁵ is hydrogen.

Compounds or prodrugs of Formula III include, but are not limited to, the following compounds:
[3-ethyl-2,6-dioxo-1-propyl-8-(1-{[3-(trifluoromethyl)phenyl]methyl}pyrazol-4-yl)-1,3,7-trihydropurin-7-yl]methyl acetate;
[3-ethyl-2,6-dioxo-1-propyl-8-(1-{[3-(trifluoromethyl)phenyl]methyl}pyrazol-4-yl)-1,3,7-trihydropurin-7-yl]methyl 2,2-dimethylpropanoate;
[3-ethyl-2,6-dioxo-1-propyl-8-(1-{[3-(trifluoromethyl)phenyl]methyl}pyrazol-4-yl)-1,3,7-trihydropurin-7-yl]methyl butanoate; and
[3-ethyl-2,6-dioxo-1-propyl-8-(1-{[3-(trifluoromethyl)phenyl]methyl}-pyrazol-4-yl)(1,3,7-trihydropurin-7-yl)]methyl dihydrogen phosphate
or pharmaceutically acceptable salts thereof

### 5. SYNTHETIC REACTION PARAMETERS

The terms “solvent,” “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present disclosure are inert organic solvents.

The term “q.s.” means adding a quantity sufficient to achieve a stated function, e.g., to bring a solution to the desired volume (i.e., 100%).

Examples of synthesis to make compounds useful in the methods of the disclosure may be found in U.S. Pat. Nos. 6,825,349; 6,997,300; 7,125,993; 7,521,554; and 7,625,881.

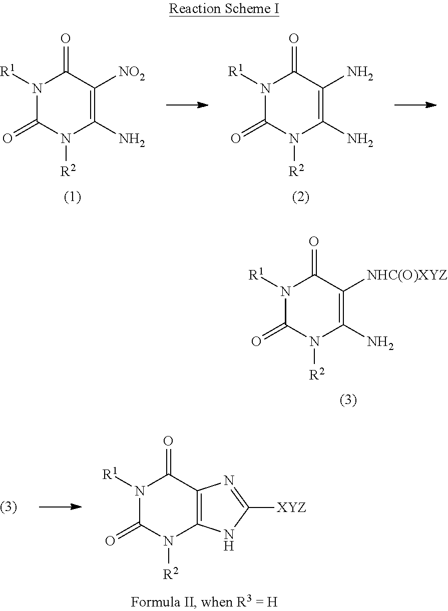

where X, Y, Z, R¹, R², and R³ are as defined above.

### Step 1—Preparation of Formula (2)

The compound of formula (2) is made from the compound of formula (1) by a reduction step. Conventional reducing techniques may be used, for example using sodium dithionite in aqueous ammonia solution; preferably reduction is carried out with hydrogen and a metal catalyst. The reaction is carried out at in an inert solvent, for example methanol, in the presence of a catalyst, for example 10% palladium on carbon catalyst, under an atmosphere of hydrogen, preferably under pressure, for example at about 30 psi, for about 2 hours. When the reaction is substantially complete, the product of formula (2) is isolated by conventional means to provide a compound of formula (2).

### Step 2—Preparation of Formula (3)

The compound of formula (2) is then reacted with a carboxylic acid of the formula Z—Y—X—CO₂H in the presence of a carbodiimide, for example 1-(3-dimethylaminopropyl)-3-ethylcarbodiimide hydrochloride. The reaction is conducted in a protic solvent, for example methanol, ethanol, propanol, and the like, preferably methanol, at a temperature of about 20-30° C., preferably about room temperature, for about 12-48 hours, preferably about 16 hours. When the reaction is substantially complete, the product of formula (3) is isolated conventionally, for example by removal of the solvent under reduced pressure, and washing the product. Alternatively, the next step can be carried out without any further purification.

### Alternative Preparation of a Compound of Formula (3)

Alternatively, the carboxylic acid of the formula Z—Y—X—CO₂H is first converted to an acid halide of the formula Z—Y—X—C(O)L, where L is chloro or bromo, by reacting with a halogenating agent, for example thionyl chloride or thionyl bromide, preferably thionyl chloride. Alternatively, oxalyl chloride, phosphorus pentachloride or phosphorus oxychloride may be used. The reaction is preferably conducted in the absence of a solvent, using excess halogenating agent, for example at a temperature of about 60-80° C., preferably about 70° C., for about 1-8 hours, preferably about 4 hours. When the reaction is substantially complete, the product of formula Z—Y—X—C(O)L is isolated conventionally, for example by removal of the excess halogenating agent under reduced pressure.

The product is then reacted with a compound of formula (2) in an inert solvent, for example acetonitrile, in the presence of a tertiary base, for example triethylamine The reaction is conducted at an initial temperature of about 0° C., and then allowed to warm to 20-30° C., preferably about room temperature, for about 12-48 hours, preferably about 16 hours. When the reaction is substantially complete, the product of formula (3) is isolated conventionally, for example by diluting the reaction mixture with water, filtering off the product, and washing the product with water followed by ether.

Step 3—Preparation of Formula II, when R³ is Hydrogen

The compound of formula (3) is then converted into a compound of Formula II by a cyclization reaction. The reaction is conducted in a protic solvent, for example methanol, ethanol, propanol, and the like, preferably methanol, in the presence of a base, for example potassium hydroxide, sodium hydroxide, sodium methoxide, sodium ethoxide, potassium t-butoxide, preferably aqueous sodium hydroxide, at a temperature of about 50-80° C., preferably about 80° C., for about 1-8 hours, preferably about 3 hours. When the reaction is substantially complete, the product of Formula II is isolated conventionally, for example by removal of the solvent under reduced pressure, acidifying the residue with an aqueous acid, filtering off the product, then washing and drying the product.

### Synthesis of the Compounds of Formula III

A method for preparing compounds of Formula I in which Y is optionally substituted lower alkyl, optionally substituted aryl, or optionally substituted heteroaryl is shown in Reaction Scheme II.

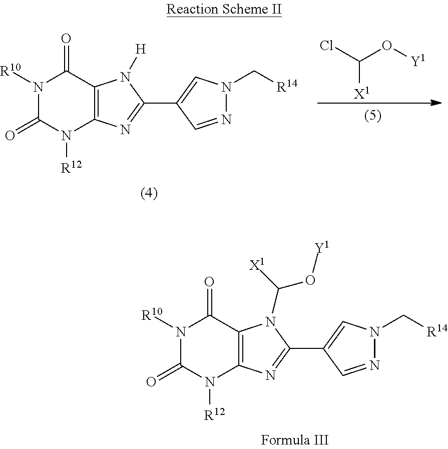

where R¹⁰, R¹², R¹⁴, X¹ and Y¹ are as defined above.

In general, the compound of formula (4) is reacted in a polar solvent, for example N,N-dimethylformamide, with a compound of formula Y¹OCHX¹Cl (5). The reaction is carried out at a temperature of about 30 to 80° C., preferably about 60° C., in the presence of a base, preferably an inorganic base, for example potassium carbonate, for about 8-24 hours. When the reaction is substantially complete, the product of Formula III is isolated by conventional means, for example preparative chromatography.

The starting compound of formula (4) can be prepared by those techniques disclosed in U.S. Pat. No. 6,825,349, or those disclosed in U.S. patent application Ser. No. 10/719,102, publication number 2004/0176399, the entire contents of which are hereby incorporated by reference.

When Y¹ is —C(O)R¹⁷, in which R¹⁷ is a heterocycle, the compound of formula (5′) (RC(O)OCHX¹Cl) is either commercially available or can be prepared as shown below, using pyridine as an example.

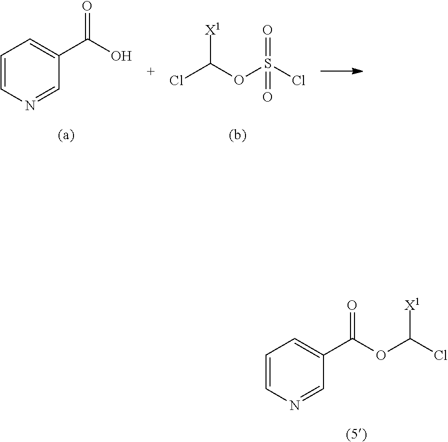

In general, the carboxylic acid of formula (a) is reacted in an inert solvent, for example dichloromethane, with a chloromethyl derivative of formula (b) in the presence of a quaternary salt, for example tetrabutylammonium sulfate. The reaction is carried out at a temperature of about 0° C., in the presence of a base, preferably an inorganic base, for example sodium bicarbonate, followed by reaction at room temperature for about 2-10 hours. When the reaction is substantially complete, the product, chloromethylpyridine-3-carboxylate (5′), is isolated by conventional means.

Carbamate derivatives can be prepared as shown in Reaction Scheme III.

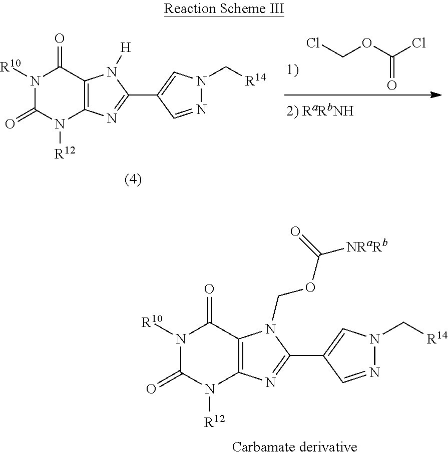

where R¹⁰, R¹² and R¹⁴, are as defined above, and R^{a}R^{b}NH represents an amine.

In general, the amine of formula R^{a}R^{b}NH is reacted in a polar solvent, for example N,N-dimethylformamide, with chloromethyl chloroformate at a temperature of about 0° C., in the presence of a base, preferably an inorganic base, for example potassium carbonate, for about 1 hour. Then a solution of the compound of formula (1) in a polar solvent at 0° C. is added, and the mixture reacted for 24 hours, allowing the temperature to rise to room temperature. When the reaction is substantially complete, the product is isolated by conventional means, for example preparative chromatography.

To prepare an ether derivative of the carbamate derivative, the derivative is reacted conventionally with an appropriate chloromethyl ether.

A method for preparing compounds of Formula III in which Y¹ is —P(O)(OH)₂ is shown in Reaction Scheme IV.

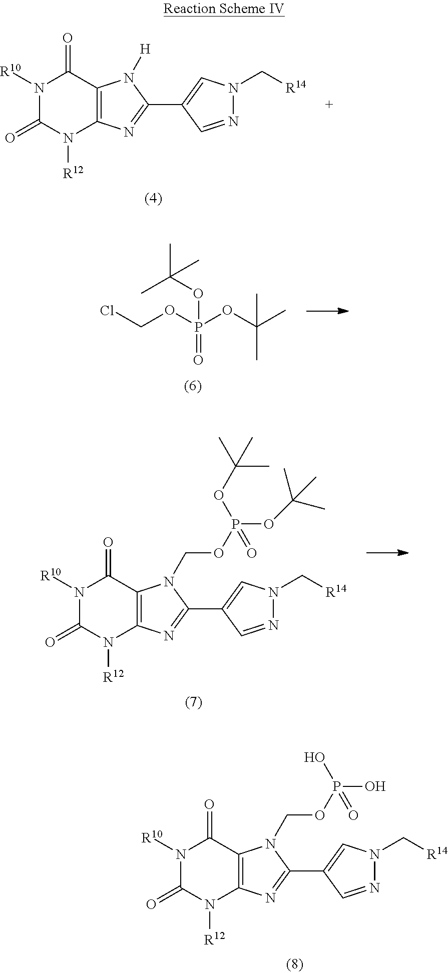

### Step 1

In general, the compound of formula (6) is reacted with a compound of formula (4) in a polar solvent, for example N,N-dimethylformamide, at a temperature of about 30-90° C., in the presence of a base, preferably an inorganic base, for example potassium carbonate, for about 4-24 hours. When the reaction is substantially complete, the product of formula (7) is isolated by conventional means and purified, for example preparative chromatography.

### Step 2

The product of formula (7) is deprotected conventionally with a strong acid, for example trifluoroacetic acid, or alternatively a weak acid such as formic acid, in an inert solvent, for example dichloromethane. The reaction is conducted at about room temperature for about 4-24 hours. When the reaction is substantially complete, the product of Formula III in which Y¹ is —P(O)(OH)₂ (8) is isolated by conventional means and purified, for example preparative chromatography.

### Starting Material of Formula (2)

The compound of formula (2), di-tert-butyl chloromethyl phosphate, is prepared from bis(tert-butoxy)phosphino-1-ol as shown below.

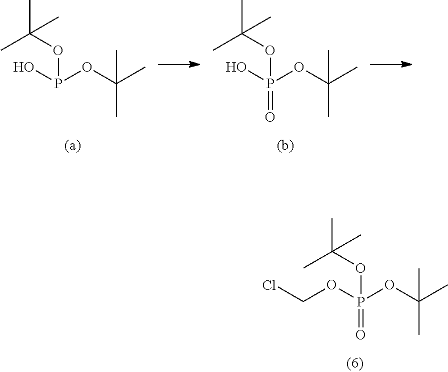

### Step 1

In general, the compound of formula (a), bis(tert-butoxy)phosphino-1-ol, is reacted with an oxidizing, for example potassium permanganate, in the presence of a mild base, for example potassium bicarbonate, in an aqueous solvent. The reaction is initially conducted at a temperature of about 0° C., and then at about room temperature for about 1 hour. When the reaction is substantially complete, the product of formula (b), ditert-butyl hydrogen phosphate, is isolated by conventional means, for example by acidification and filtration of the phosphate thus formed.

### Step 2

Initially a tetramethylammonium salt of (b) is prepared by reaction of ditert-butyl hydrogen phosphate with tetramethylammonium hydroxide in an inert solvent, for example acetone, at a temperature of about 0° C. The tetramethylammonium salt of ditert-butyl hydrogen phosphate is isolated by conventional means, for example by removal of the solvent.

The tetramethylammonium salt of ditert-butyl hydrogen phosphate is then reacted with a dihalomethane derivative, for example dibromomethane or chloroiodomethane, in an inert solvent, for example 1,2-dimethoxyethane. The reaction is conducted at a temperature of about 60-90° C. When the reaction is substantially complete, the product of formula (6) is isolated by conventional means.

### 6. COMBINATION THERAPIES

A_{2B} adenosine receptor antagonists may be administered in combination with other pulmonary hypertension therapies, including medical therapies and/or supplemental oxygen. It is contemplated that by reducing the vascular wall remodeling, the antagonists potentiate the pulmonary vasodilatory effects of current pulmonary hypertension therapies, such as calcium channel blockers, endothelin antagonists, PDE5 inhibitors, prostacyclins, and the like. Medical therapies recognized in the art to treat pulmonary hypertension include therapeutic agents, such as cardiac glycosides, vasodilators/calcium channel blockers, prostacyclins, anticoagulants, diuretics, endothelin receptor blockers, phosphodiesterase type 5 inhibitors, nitric oxide inhalation, arginine supplementation and combinations thereof.

In particular, it is contemplated that the when used in combination with endothelin receptor blockers or antagonists, including, but not limited to, ambrisentan.

Any variety of vasodilators/calcium channel blockers may used in combination with A_{2B} adenosine receptor antagonists. Examples include, but are not limited to, nifedipine, diltiazem, amlodipine, and combinations thereof.

Further, any variety of prostacyclins may be used in combination with A_{2B} adenosine receptor antagonists. Examples include, but are not limited to, epoprostenol, treprostinil, iloprost, beraprost, and combinations thereof.

In terms of administration, it is contemplated that the two or more agents can be administered simultaneously or sequentially. If the two or more agents are administered simultaneously, they may either be administered as a single dose or as separate doses. Further, it is contemplated that the attending clinician will be able to readily determine the dosage required of the additional agent, the dosing regimen, and the preferred route of administration. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3r^{d} Ed. (G. S. Banker & C. T. Rhodes, Eds.).

### 7. ADMINISTRATION

The compounds of the disclosure may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parental, particularly by injection. The forms in which the novel compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the compounds of the disclosure. Administration may be via capsule or enteric coated tablets, or the like. In making the pharmaceutical compositions that include at least one compound of the disclosure, the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds of Formula I are effective over a wide dosage range and is generally administered in a pharmaceutically effective amount. Preferably, for oral administration, each dosage unit contains from 10 mg to 2 g of a compound of the disclosure, more preferably from 10 to 700 mg, and for parenteral administration, preferably from 10 to 700 mg of a compound of the disclosure, more preferably about 50-200 mg. It will be understood, however, that the amount of the compound of the disclosure actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Formulation Example 1

Hard gelatin capsules containing the following ingredients are prepared:

| Ingredient | (mg/capsule) |
|---|---|
| Active Ingredient | 30.0 |
| Starch | 305.0 |
| Magnesium stearate | 5.0 |

The above ingredients are mixed and filled into hard gelatin capsules.

### Formulation Example 2

A tablet formula is prepared using the ingredients below:

| Ingredient | (mg/tablet) |
|---|---|
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| Ingredient | (mg/tablet) | |
|---|---|---|
| Active Ingredient | 30.0 | mg |
| Starch | 45.0 | mg |
| Microcrystalline cellulose | 35.0 | mg |
| Polyvinylpyrrolidone | | |
| (as 10% solution in sterile water) | 4.0 | mg |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Suppositories, each containing 25 mg of active ingredient are made as follows:

| Ingredient | Amount | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 6

Suspensions, each containing 50 mg of active ingredient per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 7

A subcutaneous formulation may be prepared as follows:

| Ingredient | Quantity |
|---|---|
| Active Ingredient | 5.0 mg |
| Corn Oil | 1.0 mL |

### Formulation Example 8

An injectable preparation is prepared having the following composition:

| Ingredients | Amount | |
|---|---|---|
| Active ingredient | 2.0 | mg/mL |
| Mannitol, USP | 50 | mg/mL |

| | |
|---|---|
| Gluconic acid, USP | q.s. (pH 5-6) |
| water (distilled, sterile) | q.s. to 1.0 mL |
| Nitrogen Gas, NF | q.s. |

### Formulation Example 9

A topical preparation is prepared having the following composition:

| Ingredients | grams |
|---|---|
| Active ingredient | 0.2-10 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60) C. with stirring. A sufficient quantity of water at 60) C. is then added with vigorous stirring to emulsify the ingredients, and water then added q.s. 100 g.

### EXAMPLES

The present disclosure is further defined by reference to the following examples. It will be apparent to those skilled in the art that many modifications, both to threads and methods, may be practiced without departing from the scope of the current disclosure.

### Abbreviations

Unless otherwise stated all temperatures are in degrees Celsius (° C.). Also, in these examples and elsewhere, abbreviations have the following meanings:

| | |
|---|---|
| μg = | Microgram |
| μL = | Microliter |
| μM = | Micromolar |
| ADA = | adenosine deaminase |
| AdoR = | adenosine receptor |
| BALF = | bronchoalveolar lavage fluid |
| BLM = | bleomycin |
| Comp A = | Compound A |
| DMEM = | Dulbecco Modified Eagle's Medium |
| EDTA = | ethylenediaminetetraacetic acid |
| EGM = | endothelium growth medium |
| ELISA = | enzyme-linked immunosorbent assay |
| ET-1 = | endothelin-1 |
| g = | Gram |
| HEPES = | 4-(2-hydroxyethyl)-1-piperazineethanesulfonic acid |
| HPAEC = | human pulmonary arterial endothelial cells |
| HPASM = | human pulmonary arterial smooth muscle cells |
| hr = | Hour |
| ip = | intraperitoneal |
| m = | Multiplet |
| mg = | Milligram |
| mL = | Milliliter |
| mM = | Millimolar |
| mmol = | Millimole |
| MS = | mass spectroscopy |
| NECA = | N-ethylcarboxamide adenosine |
| nM = | Nanomolar |
| NO = | nitric oxide |
| PAH = | pulmonary arterial hypertension |
| PBS = | phosphate buffered saline |
| pg = | pictograms |
| PH = | pulmonary hypertension |
| q = | Quartet |
| rpm = | revolutions per minute |
| RT-PRC = | reverse transcription-polymerase chain reaction |
| s = | Singlet |
| SM = | smooth muscle |
| SMGM = | smooth muscle growth medium |
| t = | Triplet |
| TE = | tris EDTA |
| TM = | Tris Cl, Magnesium sulfate |

### Methodologies and Reagents

### Cells and Reagents

HPASM and HPAEC and cell culture media were obtained from Lonza Group Ltd. (Base1, Switzerland). Compound A was synthesized by Gilead Sciences, Inc. (Foster City, Calif.) as discussed below in Example 1. Other chemical compounds were obtained from Sigma-Aldrich (St. Louis, Mo.).

### Cell Culture and Treatment

HPASM were grown in smooth muscle growth medium (SMGM-2). HPAECs were grown in endothelium growth medium (EGM-2). Before treatment, cells were seeded in 24-well plates and allowed to grow to ˜80% confluency. Cells were washed and then incubated in serum free basal medium in the absence or presence of adenosine receptor agonists and antagonists. In the proliferation assays, HPASM were incubated in 50% medium collected from HPAEC cells treated with vehicle or NECA.

### Real-Time RT-PCR

Gene expression was determined using real-time RT-PCR with Stratagene PCR equipment (La Jolla, Calif.). Zhong H., et al. “A_{2B} adenosine receptors increase cytokine release by bronchial smooth muscle cells, “American Journal of Respiratory Cell and Molecular Biology, 30(1): 118-125 (2004).

### Measurement of IL-6, IL-8, G-CSF, endothelin-1, and thromboxane B2

IL-6 and G-CSF were measured using human 30-plex luminex kit from Invitrogen (Carlsbad, Calif.). IL-8, endothelin-1, thromboxane B2 were measured using ELISA (kits obtained from Invitrogen, AssayDesigns (Ann Arbor, Mich.), and Caymen Biomedicals (Ann, Arbor, Mich.) respectively).

### Example 1

### Synthesis of Compound A and Prodrugs Thereof

### A. Preparation of provide 6-amino-1-ethyl-1,3-dihydropyrimidine-2,4-dione

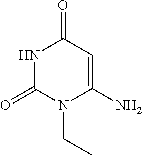

A solution of sodium ethoxide was prepared from sodium (4.8 g, 226 mmol) and dry ethanol (150 mL). To this solution was added amino-N-ethylamide (10 g, 113 mmol) and ethyl cyanoacetate (12.8 g, 113 mmol). This reaction mixture was stirred at reflux for 6 hours, cooled, and solvent removed from the reaction mixture under reduced pressure. The residue was dissolved in water (50 mL), and the pH adjusted to 7 with hydrochloric acid. The mixture was allowed to stand overnight at 0° C., and the precipitate filtered off, washed with water and air-dried, to provide 6-amino-1-ethyl-1,3-dihydropyrimidine-2,4-dione. ¹H-NMR (DMSO-d₆) δ 10.29 (s, 1H), 6.79 (s, 2H), 4.51 (s, 1H), 3.74-3.79 (m, 2H), 1.07 (t, 3H, J=7.03 Hz); MS m/z 155.98 (M⁺), 177.99 (M⁺+Na)

### . Preparation of 6-[2-(dimethylamino)-1-azavinyl]-1-ethyl-1,3-dihydropyrimidine-2,4-dione

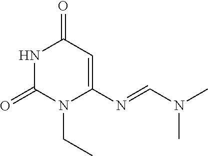

A suspension of 6-amino-1-ethyl-1,3-dihydropyrimidine-2,4-dione (0.77 g, 5 mmol) in anhydrous N,N-dimethylacetamide (25 mL) and N,N-dimethylformamide dimethylacetal (2.7 mL, 20 mmol) and was warmed at 40° C. for 90 minutes. Solvent was then removed under reduced pressure, and the residue triturated with ethanol, filtered, and washed with ethanol, to provide 6-[2-(dimethylamino)-1-azavinyl]-1-ethyl-1,3-dihydropyrimidine-2,4-dione. ¹H-NMR (DMSO-d₆) δ 10.62 (s, 1H), 8.08 (s, 1H), 4.99 (s, 1H), 3.88-3.95 (m, 2H), 3.13 (s, 3H), 2.99 (s, 3H), 1.07 (t, 3H, J=7.03 Hz); MS m/z 210.86 (M⁺), 232.87 (M⁺+Na)

### Preparation of 6-[2-(dimethylamino)-1-azavinyl]-1-ethyl-3-propyl-1,3-dihydropyrimidine-2,4-dione

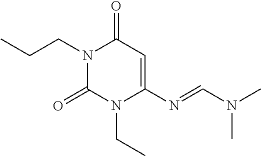

A mixture of a solution of 6-[2-(dimethylamino)-1-azavinyl]-1-ethyl-1,3-dihydropyrimidine-2,4-dione (1.5 g, 7.1 mmol) in dimethylformamide (25 mL), potassium carbonate (1.5 g, 11 mmol) and n-propyl iodide (1.54 g, 11 mmol) was stirred at 80° C. for 5 hours. The reaction mixture was cooled to room temperature, filtered, the solvents were evaporated and the product, 6-[2-(dimethylamino)-1-azavinyl]-1-ethyl-3-propyl-1,3-dihydropyrimidine-2,4-dione, was used as such in the next reaction.

### D. Preparation of 6-amino-1-ethyl-3-propyl-1,3-dihydropyrimidine-2,4-dione

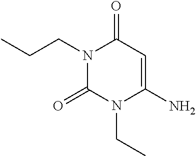

A solution of 6-[2-(dimethylamino)-1-azavinyl]-1-ethyl-3-propyl-1,3-dihydropyrimidine-2,4-dione (2.1 g) was dissolved in a mixture of methanol (10 mL) and 28% aqueous ammonia solution (20 mL), and stirred for 72 hours at room temperature. Solvent was then removed under reduced pressure, and the residue purified by chromatography on a silica gel column, eluting with a mixture of dichloromethane/methanol (15/1), to provide 6-amino-1-ethyl-3-propyl-1,3-dihydropyrimidine-2,4-dione. ¹H-NMR (DMSO-d₆) δ 6.80 (s, 2H), 4.64 (s, 1H), 3.79-3.84 (m, 2H), 3.63-3.67 (m, 2H), 1.41-1.51 (m, 2H), 1.09 (t, 3H, J=7.03 Hz), 0.80 (t, 3H, J=7.42 Hz); MS m/z 197.82 (M⁺).

### E. Preparation of 6-amino-1-ethyl-5-nitroso-3-propyl-1,3-dihydropyrimidine-2,4-dione

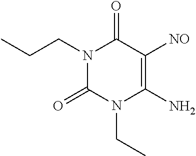

To a solution of 6-amino-1-ethyl-3-propyl-1,3-dihydropyrimidine-2,4-dione (1.4 g, 7.1 mmol) in a mixture of 50% acetic acid/water (35 mL) was added sodium nitrite (2 g, 28.4 mmol) in portions over a period of 10 minutes. The mixture was stirred at 70° C. for 1 hour, then the reaction mixture concentrated to a low volume under reduced pressure. The solid was filtered off, and washed with water, to provide 6-amino-1-ethyl-5-nitroso-3-propyl-1,3-dihydropyrimidine-2,4-dione. MS m/z 227.05 (M⁺), 249.08 (M⁺+Na)

### F. Preparation of 5,6-diamino-1-ethyl-3-propyl-1,3-dihydropyrimidine-2,4-dione

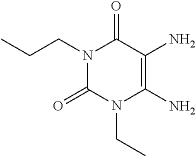

To a solution of 6-amino-1-ethyl-5-nitroso-3-propyl-1,3-dihydropyrimidine-2,4-dione (300 mg) in methanol (10 mL) was added 10% palladium on carbon catalyst (50 mg), and the mixture was hydrogenated under hydrogen at 30 psi for 2 hours. The mixture was filtered through celite, and solvent was removed from the filtrate under reduced pressure, to provide 5,6-diamino-1-ethyl-3-propyl-1,3-dihydropyrimidine-2,4-dione. MS m/z 213.03 (M⁺), 235.06 (M⁺+Na)

### F. Preparation of N-(6-amino-1-ethyl-2,4-dioxo-3-propyl(1,3-dihydropyrimidin-5-yl))(1-{[3-(trifluoromethyl)phenyl]methyl}-pyrazol-4-yl)carboxamide

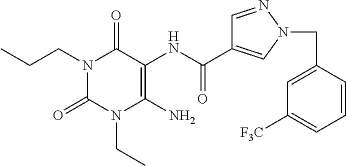

To a mixture of 5,6-diamino-1-ethyl-3-propyl-1,3-dihydropyrimidine-2,4-dione (100 mg, 0.47 mmol) and 1-{[3-(trifluoromethyl)phenyl]methyl}pyrazole-4-carboxylic acid (0.151 g, 0.56 mmol) in methanol (10 mL) was added 1-(3-dimethylaminopropyl)-3-ethylcarbodiimide hydrochloride (0.135 g, 0.7 mmol), and the reaction mixture was stirred overnight at room temperature. The solvent was removed under reduced pressure, and the residue purified using Biotage, eluting with 10% methanol/methylene chloride, to provide N-(6-amino-1-ethyl-2,4-dioxo-3-propyl(1,3-dihydropyrimidin-5-yl))(1-{[3-(trifluoromethyl)phenyl]methyl}-pyrazol-4-yl)carboxamide. ¹H-NMR (DMSO-d₆) δ 8.59 (s, 1H), 8.02 (s, 1H), 7.59-7.71 (m, 4H), 6.71 (s, 2H), 5.51 (s, 2H), 3.91-3.96 (m, 2H), 3.70-3.75 (m, 2H), 1.47-1.55 (m, 2H), 1.14 (t, 3H, J=7.03 Hz), 0.85 (t, 3H, J=7.42 Hz).

### G. Preparation of a 3-ethyl-1-propyl-8-(1-{[3-(trifluoromethyl)phenyl]methyl}pyrazol-4-yl)-1,3,7-trihydropurine-2,6-dione

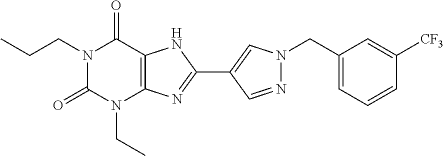

A mixture of N-(6-amino-1-ethyl-2,4-dioxo-3-propyl(1,3-dihydropyrimidin-5-yl))(1-{[3-(trifluoromethyl)phenyl]methyl}pyrazol-3-yl)carboxamide (80 mg, 0.17 mmol), 10% aqueous sodium hydroxide (5 ml), and methanol (5 ml) was stirred at 100° C. for 2 hours. The mixture was cooled, methanol removed under reduced pressure, and the residue diluted with water and acidified with hydrochloric acid. The precipitate was filtered off, washed with water, then methanol, to provide 3-ethyl-1-propyl-8-(1-{[3-(trifluoromethyl)phenyl]methyl}pyrazol-4-yl)-1,3,7-trihydropurine-2,6-dione. ¹H-NMR (DMSO-d₆) δ 8.57 (s, 1H), 8.15 (s, 1H), 7.60-7.75 (m, 4H), 5.54 (s, 2H), 4.05-4.50 (m, 2H), 3.87-3.91 (m, 2H), 1.55-1.64 (m, 2H), 1.25 (t, 3H, J=7.03 Hz), 0.90 (t, 3H, J=7.42 Hz); MS m/z 447.2 (M⁺).

H. Preparation of [3-ethyl-2,6-dioxo-1-propyl-8-(1-{[3-(trifluoromethyl)phenyl]methyl}-pyrazol-4-yl)(1,3,7-trihydropurin-7-yl)]methyl dihydrogen phosphate

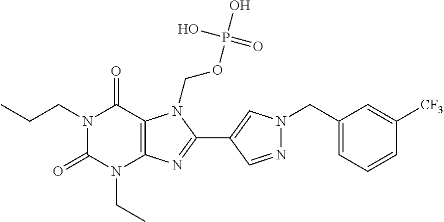

### Step 1—Preparation of di-tert-butyl chloromethyl phosphate

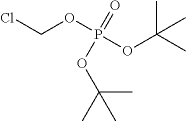

### Preparation of Ditert-Butyl Hydrogen Phosphate

To a stirred solution of bis(tert-butoxy)phosphino-1-ol (0.78 g, 4 mmol) and potassium bicarbonate (0.6 g, 2.4 mmol) in water (4 mL) at 0° C. was added (in portions) potassium permanganate (0.44 g, 2.8 mmol). The mixture was allowed to warm to room temperature, and stirred for 1 hour. Decolorizing charcoal (60 mg) was then added, and the mixture stirred at 60° C. for 15 minutes, and then filtered. The solid thus obtained was washed with water (30 mL), and the combined filtrates were treated with a further 100 mg of decolorizing charcoal at 60° C. for 20 minutes. The mixture was filtered, and the filtrate cooled to 0° C. and carefully acidified with concentrated hydrochloric acid (2 mL) with stirring. The precipitate was filtered off, washed with cold water, to provide ditert-butyl hydrogen phosphate as a white solid.

### Preparation of the Tetramethylammonium Salt of Ditert-Butyl Hydrogen Phosphate

A solution of the di-tert-butyl hydrogen phosphate obtained in step a) was dissolved in acetone (10 mL) and cooled to 0° C. To this solution was added a 10% aqueous solution of tetramethylammonium hydroxide (2.4 mL, 2.6 mmol), and the homogeneous solution was evaporated under reduced pressure to provide a solid, which was crystallized from refluxing 1,2-dimethoxyethane to provide tetramethylammonium ditert-butyl hydrogen phosphate as a white solid.

The tetramethylammonium ditert-butyl hydrogen phosphate obtained in step b was dissolved in refluxing 1,2-dimethoxymethane (15 mL), and chloroiodomethane (3.2 g, 18.1 mmol) added, and the mixture was refluxed for 90 minutes. The solvent was removed under reduced pressure, and the residue, di-tert-butyl chloromethyl phosphate, was used as such without further purification.

### Step 2

A solution of 3-ethyl-1-propyl-8-(1-{[3-(trifluoromethyl)phenyl]methyl}-pyrazol-4-yl)-1,3,7-trihydropurine-2,6-dione (0.47 g, 1 mmol) was dissolved in 20 mL of N,N-dimethylformamide, and potassium carbonate (0.42 g, 4 mmol) was added, followed by di-tert-butyl chloromethyl phosphate (0.34 g, 1.32 mmol), and the mixture was stirred at 60° C. overnight. The reaction mixture was cooled, and the precipitate filtered off, washing with ethyl acetate. The filtrate was concentrated under reduced pressure, and the residue was purified by preparative thin layer chromatography, eluting with 4% methanol/methylene chloride, to provide tert-butyl [3-ethyl-2,6-dioxo-1-propyl-8-(1-{[3-(trifluoromethyl)phenyl]methyl}pyrazol-4-yl)(1,3,7-trihydropurin-7-yl)]methyl methylethyl phosphate (0.26 g) as a colorless oil.

### Step 3

A solution of tert-butyl [3-ethyl-2,6-dioxo-1-propyl-8-(1-{[3-(trifluoromethyl)phenyl]methyl}pyrazol-4-yl)(1,3,7-trihydropurin-7-yl)]methyl methylethyl phosphate (80 mg, 0.12 mmol) was dissolved in methylene chloride (6 mL) and trifluoroacetic acid (0.72 mmol) was added. The mixture was stirred at room temperature overnight. The solvent was removed under reduced pressure, and the solid white residue was triturated with ether and collected by filtration, providing [3-ethyl-2,6-dioxo-1-propyl-8-(1-{[3-(trifluoromethyl)phenyl]methyl}-pyrazol-4-yl) (1,3,7-trihydropurin-7-yl)]methyl dihydrogen phosphate (41 mg).

NMR¹H-NMR (DMSO-d₆) δ 8.70 (s, 1H), 8.15 (s, 1H), 7.74 (s, 1H), 7.69-7.71 (m, 1H), 7.60-7.63 (m, 2H), 6.12 (d, 2H, J=5.4 Hz), 5.54 (s, 2H), 4.06 (q, 2H, J=13.8 Hz), 3.84 (t, 2H, J=7.4 Hz), 1.52-1.62 (m, 2H), 1.25 (t, 3H, J=7.0 Hz), 0.87 (t, 3H, J=7.4 Hz); MS m/z 579.02 (M⁺+Na)

### Example 2

### Adenosine Receptor Assays

In order to screen for A_{2B} antagonist, two type of assays are typically used: 1) radioligand binding assay to determine that a given compound could bind to A_{2B} receptor as described below and 2) a functional assay (cAMP assay or others) to determine whether the compound is an agonist (activates the receptor) or an antagonist (inhibits the activation of the receptor).

A radioligand binding assay for A_{2B} adenosine receptor is used to determine the affinity of a compound for the A2B adenosine receptor. Meanwhile, the radioligand binding assays for other adenosine receptors are conducted to determine affinities of the compound for A₁, A_{2A} and A₃ adenosine receptors. The compound should have a higher affinity (at least 3 fold) for A_{2B} receptor than other adenosine receptors.

A cAMP assay for A_{2B} receptor is often used to confirm that the compound is an antagonist and will blocks the A_{2B} receptor-mediated increase in cAMP.

### Radioligand Binding for A_{2B} Adenosine Receptor

Compounds that are putative antagonists of the A_{2B} receptor may be screened for requisite activity based on the following assays. Human A_{2B} adenosine receptor cDNA are stably transfected into HEK-293 cells (referred to as HEK-A2B cells). Monolayer of HEK-A2B cells are washed with PBS once and harvested in a buffer containing 10 mM HEPES (pH 7.4), 10 mM EDTA and protease inhibitors. These cells are homogenized in polytron for 1 minute at setting 4 and centrifuged at 29000 g for 15 minutes at 4° C. The cell pellets are washed once with a buffer containing 10 mM HEPES (pH 7.4), 1 mM EDTA and protease inhibitors, and are resuspended in the same buffer supplemented with 10% sucrose. Frozen aliquots are kept at −80° C. Competition assays are started by mixing 10 nM ³H-ZM241385 (Tocris Cookson) with various concentrations of test compounds and 50 μg membrane proteins in TE buffer (50 mM Tris and 1 mM EDTA) supplemented with 1 Unit/mL adenosine deaminase. The assays are incubated for 90 minutes, stopped by filtration using Packard Harvester and washed four times with ice-cold TM buffer (10 mM Tris, 1 mM MgCl₂, pH 7.4). Non specific binding is determined in the presence of 10 μM ZM241385. The affinities of compounds (I.e. Ki values) are calculated using GraphPad software.

### Radioligand Binding for Other Adenosine Receptors

Human A₁, A_{2A}, A₃ adenosine receptor cDNAs are stably transfected into either CHO or HEK-293 cells (referred to as CHO-A1 HEK-A2A, CHO-A3). Membranes are prepared from these cells using the same protocol as described above. Competition assays are started by mixing 0.5 nM ³H—CPX (for CHO-A1), 2 nM ³H-ZM241385 (HEK-A2A) or 0.1 nM ¹²⁵I-AB-MECA (CHO-A3) with various concentrations of test compounds and the perspective membranes in TE buffer (50 mM Tris and 1 mM EDTA of CHO-A1 and HEK-A2A) or TEM buffer (50 mM Tris, 1 mM EDTA and 10 mM MgCl₂ for CHO-A3) supplemented with 1 Unit/mL adenosine deaminase. The assays are incubated for 90 minutes, stopped by filtration using Packard Harvester and washed four times with ice-cold TM buffer (10 mM Tris, 1 mM MgCl₂, pH 7.4). Non specific binding is determined in the presence of 1 μM CPX (CHO-A1), 1 μM ZM214385 (HEK-A2A) and 1 μM IB-MECA (CHO-A3). The affinities of compounds (I.e. Ki values) are calculated using GraphPad software.

### cAMP Measurements

Monolayer of transfected cells are collected in PBS containing 5 mM EDTA. Cells are washed once with DMEM and resuspended in DMEM containing 1 Unit/mL adenosine deaminase at a density of 100,000 500,000 cells/mL. 100 μL of the cell suspension is mixed with 25 μL containing various agonists and/or antagonists and the reaction was kept at 37° C. for 15 minutes. At the end of 15 minutes, 125 μL 0.2N HCl is added to stop the reaction. Cells are centrifuged for 10 minutes at 1000 rpm. 100 μL of the supernatant is removed and acetylated. The concentrations of cAMP in the supernatants are measured using the direct cAMP assay from Assay Design.

A_{2A} and A_{2B} adenosine receptors are coupled to Gs proteins and thus agonists for A_{2A} adenosine receptor (such as CGS21680, CAS# 20225-54-9) or for A_{2B} adenosine receptor (such as NECA) increase the cAMP accumulations whereas the antagonists to these receptors prevent the increase in cAMP accumulations-induced by the agonists. A₁ and A₃ adenosine receptors are coupled to Gi proteins and thus agonists for A₁ adenosine receptor (such as CPA) or for A₃ adenosine receptor (such as IB-MECA) inhibit the increase in cAMP accumulations-induced by forskolin. Antagonists to A₁ and A₃ receptors prevent the inhibition in cAMP accumulations.

It is within the skill of one in the art to determine if a compound, based on the above assay protocol, is an antagonist of the A_{2B} receptor antagonist.

### Example 3

### Expression of Adenosine Receptors in HPASM and HPAEC

This example shows that among four subtypes of adenosine receptors (A₁, A_{2A}, A_{2B}, and A₃), A_{2B} has the highest expression in human pulmonary arterial cells.

Expression of four subtypes, A₁, A_{2A}, A_{2B}, and A₃, of adenosine receptors in human pulmonary arterial endothelial cells (HPAEC) and human pulmonary arterial smooth muscle cells (HPASM) was determined using quantitive real-time RT-PCR using the methodologies described above.

The results are presented in FIG. 1 (HPAEC) and FIG. 2 (HPASM). As can be seen in the figures, in both cell types, the A_{2B} expression was surprisingly the highest among the four subtypes of AdoRs as shown in terms of percentage of β-actin. Expression of A₁ and A₃ was not detected in either cells.

### Example 4

### Bleomycin-Induced Vascular Wall-Thickening is Mediated by A_{2b} Receptor

This example shows the role of A_{2B} receptor in bleomycin-induced vascular wall-thickening and thus demonstrates its involvement the pathogenesis of pulmonary hypertension.

Bleomycin is a glycopeptide antibiotic produced by the bacterium Streptomyces verticillus. It is a known anticancer agent with associated serious complications that include pulmonary fibrosis and impaired lung function. It has been suggested that bleomycin induces sensitivity to oxygen toxicity and recent studies support the role of the proinflammatory cytokines IL-18 and IL-1beta in the mechanism of bleomycin-induced lung injury.

FIG. 4A-I show the vascular changes in wild type and A_{2B} receptor knockout (KO) mice exposed to bleomycin. Mice were subjected to an intraperitoneal injection of bleomycin (0.35 units) or saline every 4 days for 33 days. At the end of the protocol, lungs were processed for H&E staining FIGS. 4A, 4D, and 4G show the distal arteries, proximal arteries, and preacinar pulmonary arteries, respectively, from wild type mice exposed to saline. FIGS. 4B, 4E and 4H show the distal arteries, proximal arteries, and preacinar pulmonary arteries, respectively, from wild type mice exposed to bleomycin. FIGS. 4C, 4F and 4I show the distal arteries, proximal arteries, and preacinar pulmonary arteries, respectively, from A_{2B} receptor KO mice exposed to bleomycin. Wild type mice exposed to bleomycin showed increased muscularity around the small distal pulmonary arteries and more proximal pulmonary arteries, suggesting that these mice had classical morphological features of pulmonary hypertension. Interestingly, the A_{2B} receptor KO mice exposed to bleomycin did not exhibit these vascular changes suggesting that the A_{2B} receptor is involved in the pathogenesis of pulmonary hypertension.

### Example 5

### Release of IL-8 in Endothelial Cells

This example shows that activation of A_{2B} receptor induces the release of IL-8, and the induction can be inhibited by A_{2B} adenosine receptor antagonists.

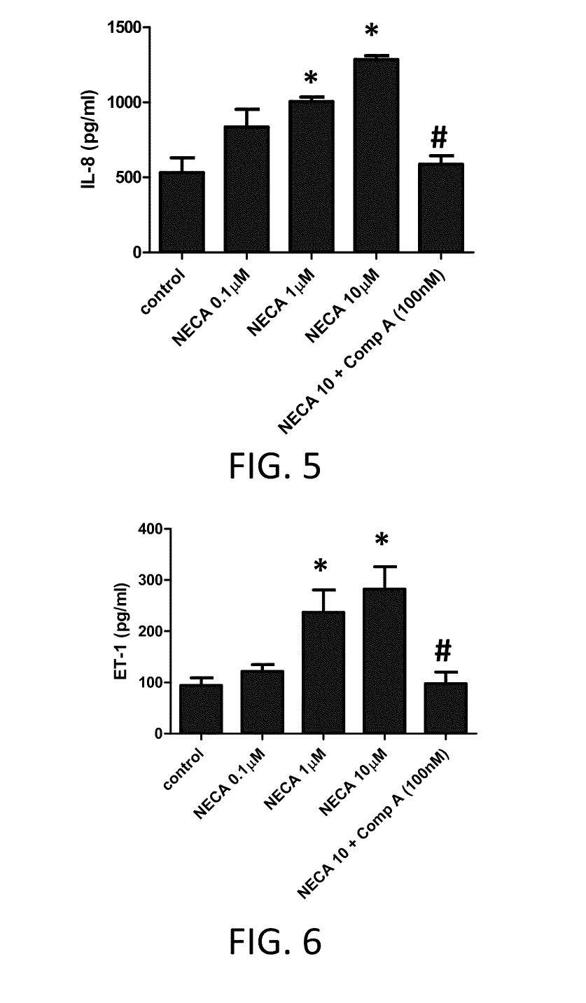

HPAECs were incubated in basal medium in the absence or presence of NECA (N-ethylcarboxamide adenosine) at various concentrations (0.1 μM, 1 μM, and 10 μM) and Compound A (100 nM) for 18 hours. NECA is a known adenosine agonist of A₁ and A₂ subtypes. The amount of IL-8, provided in pg/mL, was measured by ELISA. Results are presented in FIG. 5. As can be seen in FIG. 5, NECA dose-dependently increased the release of IL-8 at 18 hr. This effect of NECA (10 μM) was markedly reduced by A_{2B} adenosine receptor antagonist, Compound A (Comp A), suggesting that the activation of A_{2B} receptor induced the release of IL-8.

### Example 6

### Endothelin-1 Release from HPAECs

Similar to Example 5, this example shows that activation of A_{2B} receptor induces the release of ET-1, and the induction can be inhibited by A_{2B} adenosine receptor antagonists.

HPAECs were incubated in the absence or presence of NECA at various concentrations (0.1 μM, 1 μM, and 10 μM) and Compound A (100 nM) for 18 hours. The amount of ET-1, provided in pg/mL was measured by the ELISA protocol discussed above. The results are presented in FIG. 6. As can be seen in FIG. 6, NECA dose-dependently increased the release of ET-1 at 18 hr. This effect of NECA (10 μM) was markedly reduced by A_{2B} adenosine receptor antagonist, Compound A, suggesting that the activation of A_{2B} receptor induced the release of ET-1.

### Example 7

### Cytokine Release from HPASMs

Similar to Examples 5 and 6, this example shows that activation of A_{2B} receptor induces the release of cytokines in muscle cells as well, which can be inhibited by A_{2B} adenosine receptor antagonists.

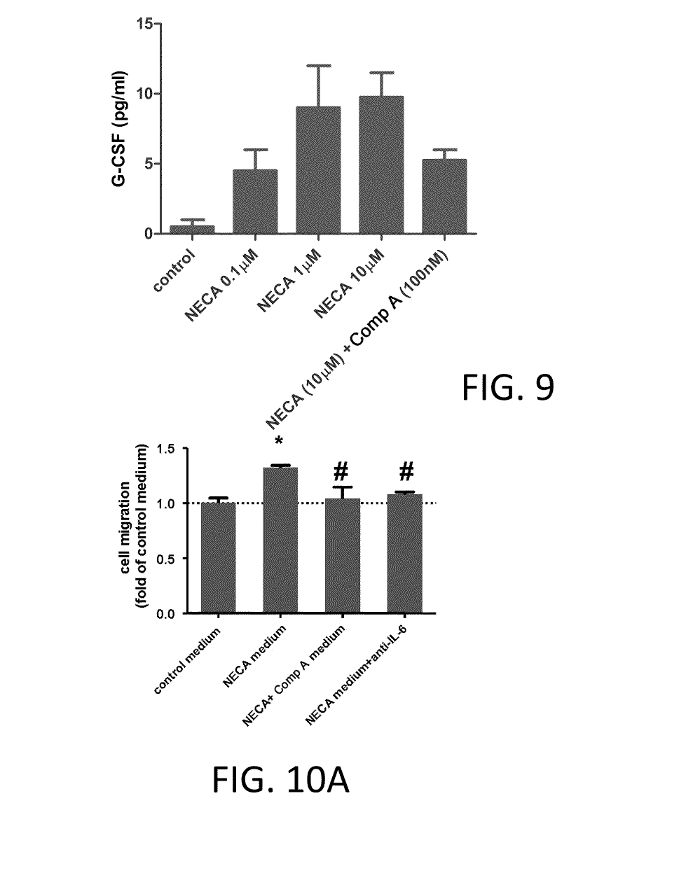

HPASMs were incubated in the absence or presence of NECA at various concentrations (0.1 μM, 1 μM, and 10 μM) and Compound A (100 nM) for 18 hours. NECA dose-dependently increased the release of IL-6 (see, FIG. 7), IL-8 (see, FIG. 8) and G-CSF (FIG. 9) at 18 hr. These effects of NECA (10 μM) were markedly reduced by A_{2B} adenosine receptor antagonist, Compound A, suggesting that the activation of A_{2B} receptor induced the release of these cytokines.

### Example 8

### Smooth Muscle Cell Migration

This example shows that NECA increases smooth muscle migration and the increase can be inhibited by the A_{2B} adenosine receptor antagonist, Compound A or anti-IL-6 antibody.

Conditional media were collected from HPASMs treated with vehicle, NECA (10 μM), NECA (10 μM) and Compound A (100 nM), or NECA (10 μM) and an anti-IL-6 antibody (1 ng/mL, purchased from Invitrogen) for 18 hours were added to the lower wells of Boyden chamber assay systems as chemoattractants. HPASMs were allowed to migrate for 24 hours. As shown in FIG. 10A, NECA increased smooth muscle cell migration and the incease was inhibited by either Compound A or the anti-IL-6 antibody. It was also observed that IL-8 neutralizing antibody had no effect on cell migration. Therefore, this example indicates that through activating A_{2B} adenosine receptor, NECA activates smooth muscle which releases IL-6. The released IL-6 in turn enhances smooth muscle cell migration (see FIG. 10B for illustration).

### Example 9

### Thromboxane B2 Release from HPASMs

This example shows that, in HPASMs, the activation of A_{2B} receptor induces the release of thromboxane B2, which is known to induce pulmonary vasoconstriction.

HPASMs were incubated in the absence or presence of NECA at various concentrations (0.1 μM, 1 μM, and 10 μM) and Compound A (100 nM) for 18 hours. As can be seen in FIG. 11, NECA dose-dependently increased the release of thromboxane B2 at hour 18. This effect of NECA (10 μM) was markedly reduced by Compound A, suggesting that the activation of A_{2B} receptor induced the release of thromboxane B2.

### Example 10

### Expression of Collagen, Other Extracellular Matrix Proteins, and Extracellular Matrix Enzymes

HPASMs were incubated in the presence of NECA (10 μM) or NECA (10 μM) together with Compound A (100 nM) for 1.5 hours. A real-time-RT-PCR array focusing on genes involved in tissue remodeling were conducted on the RNAs isolated from the HPASMs. NECA increased the mRNA expression of ADAMTS1, ADAMTS8, CDH1, MMPI, MMP12, HAS1, ITGA7, COL1A1, COL8A1 and CTGF (FIG. 12A-B). These effects of NECA were reduced by Compound A (FIG. 12C), suggesting that the activation of A_{2B} receptor induced the release of these genes.

### Example 11

### Effect of NECA-Activated HPAECs on Proliferation of HPASMs

This example shows that A_{2B} receptors in HPAECs increase the release of ET-1 which in turn induce proliferation of the HPASMs. Treatment with an A_{2B} adenosine receptor antagonist, on the other hand, inhibits such induction.

Cell supernatants were collected from HPAECs treated with vehicle (control medium), NECA (10 μM, NECA medium) or NECA and Compound A (100 nM) for 18 hours. These cell supernatants (diluted 1:1 in Murashige and Skoog (MS) basal medium) with or without ambrisentan (30 nM) were used to incubate HPASMs for 18 hours. Cells were counted. The results are presented in FIG. 13A. NECA-HPAEC medium increased cell number of HPASMs at 18 hours compared to control-HPAEC medium. This finding suggests that certain mediator induced by NECA and released from HPAEC may be able to promote proliferation of HPASM or prevent cell death of HPASM.

As shown in FIG. 13A, treatment with both Compound A and ambrisentan inhibited the NECA induced proliferation. Specifically, the data demonstrate that Compound A (available from Gilead Sciences, Inc.) inhibits the activation of endothelial cells, which in turn, decreases the release of ET-1. Ambrisentan, an antagonist of the ETA (endothelin A) receptor, inhibits the proliferation of HPASM induced by NECA activated HPASMs. Therefore, adenosine activated HPAECs are able to induce proliferation of the HPASMs, and this is mediated by A_{2B} receptors in HPAECs that lead to increased release of ET-1.

### Example 12

### NECA-Induced Expression of NOTCH3 in HPASMs

It is contemplated that pulmonary hypertension may be characterized by an overexpression of NOTCH3 in small pulmonary artery smooth muscle cells. Further, the severity of the disease may also be correlated with the amount of NOTCH3 protein in the lung. See, Li, X., et al., “Notch3 signaling promotes the development of pulmonary arterial hypertension” Nature Medicine, 15(11):1289-1297 (2009).

HPASMs were incubated with NECA (10 μM) or NECA (10 μM) along with Compound A (100 nM) for 1.5 hours. The expression of NOTCH3 was measured by quantitive real-time RT-PCR using the methodologies described above.

The results are presented in FIG. 14. As can be seen in the figure, gene expression of NOTCH3 and the increase of NOTCH3 expression induced by NECA were inhibited by Compound A. Therefore, it is further contemplated that pulmonary hypertension may be treated using an A_{2B} adenosine receptor antagonist.

### Example 13

### Attenuation of Vascular Wall Thickening in the Lungs of ADA-Deficient Mice

This example demonstrates that treatment with A_{2B} adenosine receptor antagonists attenuates thichening of vascular wall in an adenosine-dependent pulmonary injury model.

The model system being used is the adenosine deaminase (ADA)-deficient mouse model of adenosine-dependent pulmonary injury. The mice were obtained according to the method described in Blackburn, M. et al. “Adenosine Deaminase-deficient Mice Generated Using a Two-stage Genetic Engineering Strategy Exhibit a Combined Immunodeficiency” J. Biol. Chem., 273(9):5093-5100 (1998).

This example follows the protocol described in Sun C X, et al. “Role of A_{2B} adenosine receptor signaling in adenosine-dependent pulmonary inflammation and injury,” J. Clin. Invest., 116(8):2173-2182 (2006), which is hereby incorporated by reference.

All ADA-deficient mice were maintained on ADA enzyme therapy from birth to postnatal day 21 to prevent defects in alveolar development. Banerjee, et al. Am. J. Respir. Cell Mol. Biol. 30-38-50 (2004). ADA enzyme therapy was discontinued at postnatal day 21, and 3 days later the mice were given intraperitoneal injections of 1 mg/kg of Compound A twice daily for 14 days.

Lungs were collected from postnatal day 38 mice and prepared routinely for sectioning and H&E staining Tissues were taken from control (ADA+) mice (FIG. 3A), the ADA-deficient mice (FIG. 3B), and the ADA-deficient mouse treated with Compound A (FIG. 3C). Sections are representative of 6-8 different mice from each treatment group. As can be seen in the figures, the ADA−/− mice showed an increase in vascular wall thickening compared to that of the ADA+ mice. Further, the thickening in the ADA−/− mouse treated with Compound A is drastically reduced.

### Example 14

### Adenosine A_{2B} Receptor Modulates Pulmonary Hypertension Associated with Chronic Lung Disease

This example illuminates the role of A_{2B} adenosine receptor in the pathogenesis of pulmonary hypertension associated with chronic lung injury and demonstrates that an A_{2B} adenosine receptor antagonist is useful in treating such pulmonary hypertension.

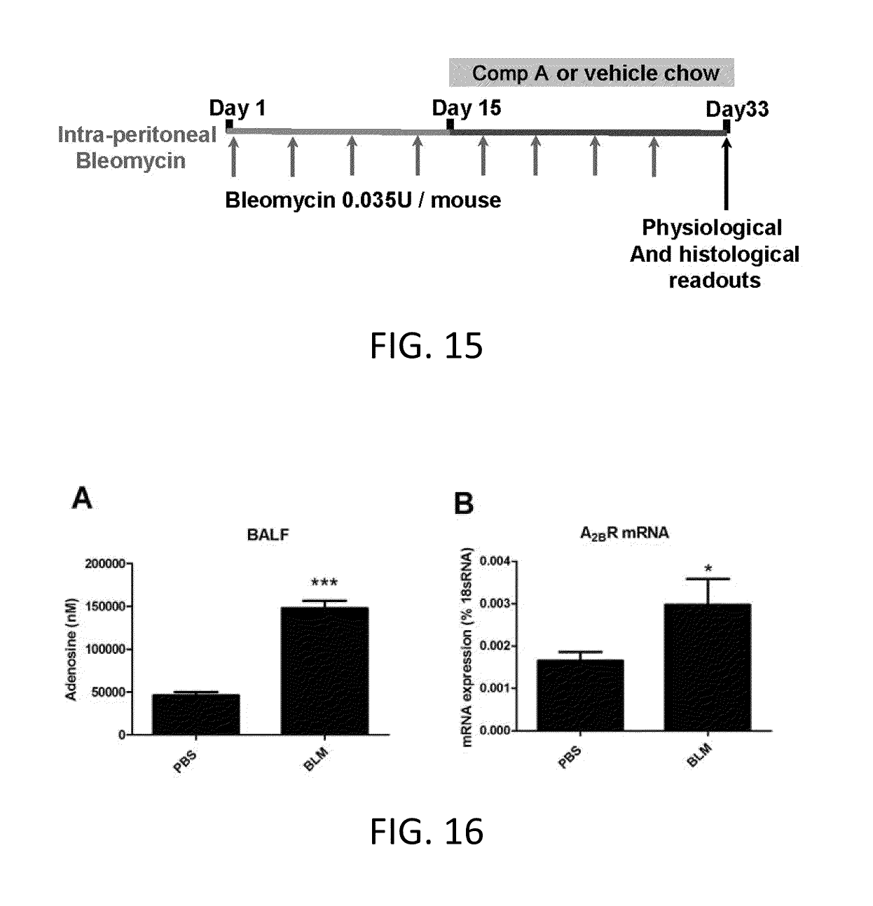

Methods: Male C57BL6 mice were treated with bleomycin (BLM) at 0.035 units per mouse, or vehicle (phosphate buffered saline (PBS)) intra-peritoneally twice weekly for 4 weeks. When pulmonary fibrosis was established, on day 15, mice were provided with special chow containing an A2B receptor antagonist, Compound A (˜10 mg/kg/day dose), for the next 18 days (FIG. 15). In contrast, control groups received normal chow.

On day 33, right ventricle systolic pressure (RVSP), systemic blood pressure, heart rate and lung function measurements were performed. Additionally, the lungs were collected for immunohistochemistry (IHC) for α-smooth muscle actin (αSMA).

Statistical Analysis: All data were analyzed using a 1-way ANOVA with a Newman-Keuls post test. The software used to conduct the statistical analysis was Graph-Pad Prism v5.00 (La Jolla Calif.). In all related figures, significance levels: *P<0.05, **0M01<P<0.01, ***P<0.001 refer to comparisons between PBS and BLM groups; significance levels: #P<0.05, # # 0.001<P<0.01, # # # P<0.001 refer to comparisons between BLM and BLM+Compound A groups. All values in the figures represent mean±SEM (standard error or the mean) for 5-8 mice per group.

Results: Pulmonary hypertension (PH) is often associated with underlying chronic lung diseases such as chronic obstructive pulmonary disease (COPD) and pulmonary fibrosis. In some classification systems, PH is classified into five groups and PH associated with lung diseases is classified as Group 3 (e.g., Simonneau et al., “Updated Clinical Classification of Pulmonary Hypertension,” J Am Coll Cardiol 54:S43-54 (2009)).

Here, a pulmonary fibrosis animal model is established with treatment with bleomycin (BLM). As described above, BLM is a glycopeptide antibiotic produced by the bacterium Streptomyces verticillus, which is a known anticancer agent with associated serious complication that includes pulmonary fibrosis and impaired lung function. As shown in FIG. 16A-B, adenosine levels, measured by HPLC, from bronchoalveolar lavage fluid (BALF) of mice, and A_{2B}R expression levels from fresh frozen lungs increased significantly following bleomycin treatment.

Changes of vascular remodeling following bleomycin exposure and the effects of Compound A. were evident from FIG. 17A, showing immunostaining for α-SMA to identify myofibroblasts (gray signal) in the parenchyma (upper panels) and the muscular wall of vessels (arrows and lower panels). BLM significantly increased the extent of vascular muscularization (FIG. 17B) and the number of muscularized vessels (FIG. 17C) which increases were attenuated in Compound A-treated mice or A_{2B}R^{-/-} nice. Further, as shown in FIG. 18, BLM significantly increased RVSP (left panel) and RV hypertrophy (right panel). Such increases, however, were also attenuated in Compound A-treated mice or A_{2B}R^{-/-} mice. Additionally, BLM increased peri-vascular fibrosis as indicated by total collagen levels in the lung, which increase was likewise attenuated in Compound A-treated mice or A_{2B}R^{-/-} mice (FIG. 19).

FIG. 20A-B include a number of lung function measurements showing the effects of bleomycin treatment and Compound A. In all instances, BLM had a significant impace on the lung function (e.g., increased dynamic resistance of the lungs (A), increased tissue damping (B), increased quasi-static elastance (C) and decreased arterial oxygenation levels (D). All such effects, however, were attenuated by the treatment of Compound A or in the A_{2B}R^{-/-} mice.

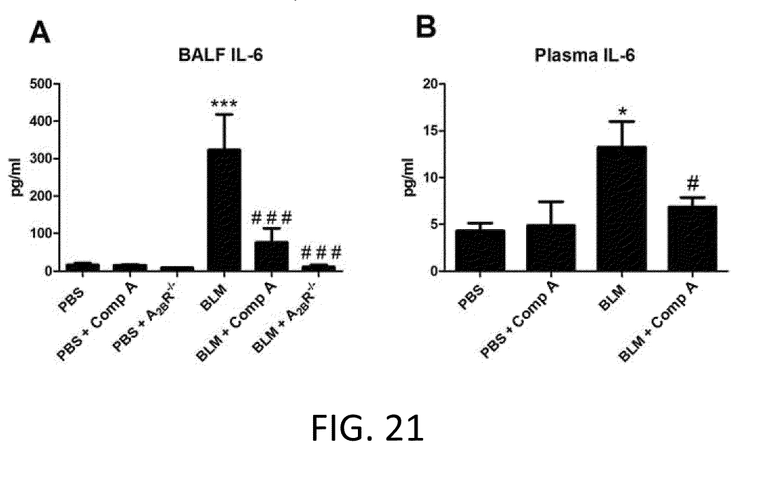

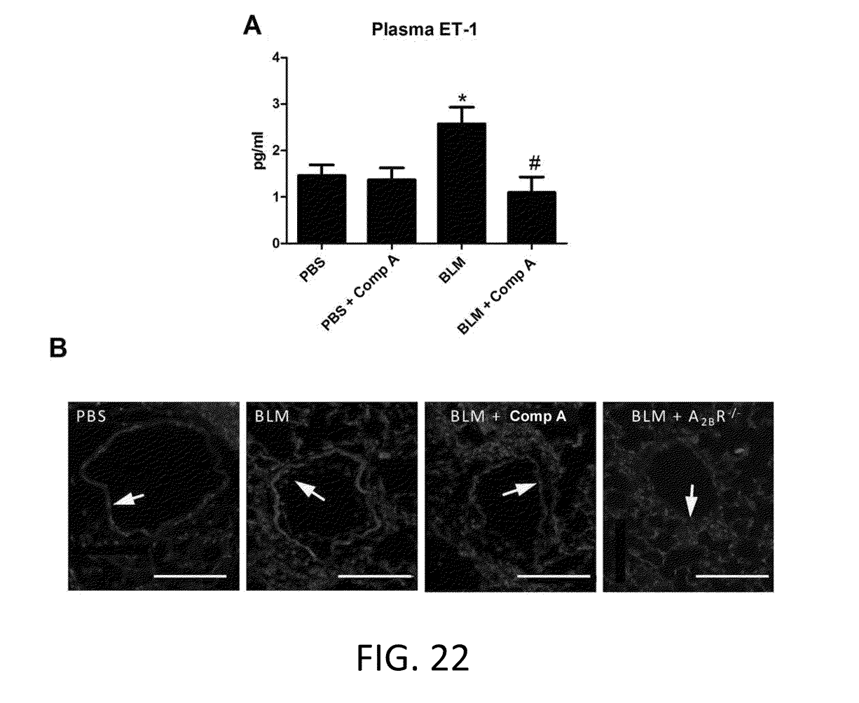

Similar to Exmples 7 and 8, in the BLM PH animal model, BLM significantly increased the release of interleukin (IL)-6 level (FIG. 21) and ET-1 (FIG. 22), and consistent with the above observations, such increases were significantly attenuated by the treatment of Compound A or in the A_{2B}R^{-/-} mice.

In summary, mice exposed to BLM had increased RVSP compared to control mice. No changes in systemic systolic blood pressure or heart rate were observed between the treatment groups. Measurements of lung functions revealed increased airway resistance and a reduction in airway and tissue compliance, in BLM-exposed mice, consistent with the development of pulmonary fibrosis. IHC for αSMA exhibited an increase in neo-muscularized vessels following BLM exposure. Blockade of the A_{2B} receptor was able to inhibit BLM-induced increase in RVSP as well as attenuating the effects of BLM in lung functions and reducing the extent of pulmonary vessel muscularization.

These results highlight the role of the A_{2B} receptor in the pathogenesis of pulmonary hypertension associated with chronic lung injury and confirm the A_{2B} receptor as a valid target for the treatment of pulmonary hypertension.

It will be appreciated that those skilled in the art will be able to devise various arrangements which, although not explicitly described or shown herein, embody the principles of the disclosure and are included within its spirit and scope. Furthermore, all conditional language recited herein is principally intended to aid the reader in understanding the principles of the disclosure and the concepts contributed by the inventors to furthering the art, and are to be construed as being without limitation to such specifically recited conditions. Moreover, all statements herein reciting principles, aspects, and embodiments of the disclosure are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

What is claimed is:

1. A method of treating pulmonary hypertension in a patient in need thereof, said method comprising administering to the patient a therapeutically effective amount of an A_{2B} adenosine receptor antagonist.

2. The method of claim 1, wherein the pulmonary hypertension is pulmonary arterial hypertension (PAH).

3. The method of claim 2, wherein the pulmonary arterial hypertension is selected from idiopathic PAH, familial PAH, or PAH associated with another disease or condition.

4. The method of claim 1, wherein the method is for the treatment of pulmonary inflammation.

5. The method of claim 1, wherein the pulmonary hypertension is pulmonary hypertension owing to lung diseases and/or hypoxia.

6. The method of claim 1, wherein the patient is human.

7. The method of claim 1, wherein the administration is systemic.

8. The method of claim 1, wherein the administration is oral.

9. The method of claim 1, wherein the administration is intravenous.

10. The method of claim 1, wherein the administration is intramuscular.

11. The method of claim 1, wherein the administration is intraperitoneal.

12. The method of claim 1, wherein the administration is by inhalation.

13. The method of claim 1, wherein the A_{2B} receptor antagonist is a 8-cyclic xanthine derivative.

14. The method of claim 1, wherein the A_{2B} receptor adenosine antagonist is a compound of Formula I or II: 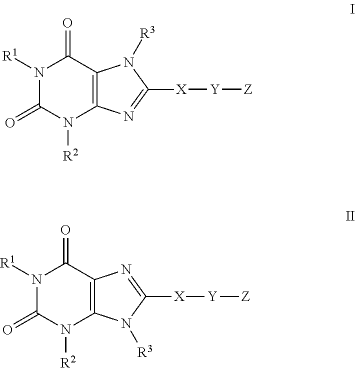
wherein:
R¹ and R² are independently chosen from hydrogen, optionally substituted alkyl, or a group -D-E, in which D is a covalent bond or alkylene, and E is optionally substituted alkoxy, optionally substituted cycloalkyl, optionally substituted aryl, optionally substituted heteroaryl, optionally substituted heterocyclyl, optionally substituted alkenyl or optionally substituted alkynyl, with the proviso that when D is a covalent bond E cannot be alkoxy;
R³ is hydrogen, optionally substituted alkyl or optionally substituted cycloalkyl;
X is optionally substituted arylene or optionally substituted heteroarylene;
Y is a covalent bond or alkylene in which one carbon atom can be optionally replaced by —O—, —S—, or —NH—, and is optionally substituted by hydroxy, alkoxy, optionally substituted amino, or —COR¹⁶, in which R¹⁶ is hydroxy, alkoxy or amino;
with the proviso that when the optional substitution is hydroxy or amino it cannot be adjacent to a heteroatom; and
Z is optionally substituted monocyclic aryl or optionally substituted monocyclic heteroaryl; or
Z is hydrogen when X is optionally substituted heteroarylene and Y is a covalent bond;
with the proviso that when X is optionally substituted arylene, Z is optionally substituted monocyclic heteroaryl
or a pharmaceutically acceptable salt, tautomer, isomer, a mixture of isomers, or prodrug thereof.

15. The method of claim 14, wherein R¹ and R² are independently hydrogen, optionally substituted lower alkyl, or a group -D-E, in which D is a covalent bond or alkylene, and E is optionally substituted phenyl, optionally substituted cycloalkyl, optionally substituted alkenyl, or optionally substituted alkynyl.

16. The method of claim 14, wherein R³ is hydrogen.

17. The method of claim 14, wherein R¹ and R² are independently lower alkyl optionally substituted by cycloalkyl and X is optionally substituted phenylene.

18. The method of claim 17, wherein Y is alkylene where a carbon atom is replaced by oxygen.

19. The method of claim 18, wherein Y is —O—CH₂— and the oxygen is the point of attachment to phenylene.

20. The method of claim 19, wherein Z is optionally substituted oxadiazole.

21. The method of claim 20, wherein Z is optionally substituted [1,2,4]-oxadiazol-3-yl with optionally substituted phenyl or by optionally substituted pyridyl.

22. The method of claim 14, wherein X is optionally substituted 1,4-pyrazolene.

23. The method of claim 22, wherein Y is a covalent bond, alkylene, lower alkylene, and Z is hydrogen, optionally substituted phenyl, optionally substituted pyridyl or optionally substituted oxadiazole.

24. The method of claim 23, wherein R¹ is lower alkyl optionally substituted by cycloalkyl, and R² is hydrogen.

25. The method of claim 22, wherein Y is —(CH₂)— or —CH(CH₃)— and Z is optionally substituted phenyl, or Y is —(CH₂)— or —CH(CH₃)— and Z is optionally substituted oxadiazole, particularly 3,5-[1,2,4]-oxadiazole, or Y is —(CH₂)— or —CH(CH₃)— and Z is optionally substituted pyridyl.

26. The method of claim 25, wherein R¹ and R² are independently lower alkyl optionally substituted by cycloalkyl.

27. The method of claim 22, wherein Y is a covalent bond, —(CH₂)— or —CH(CH₃)— and Z is hydrogen, optionally substituted phenyl, or optionally substituted pyridyl.

28. The method of claim 27, wherein Y is a covalent bond and Z is hydrogen.

29. The method of claim 1, wherein the receptor antagonist is selected from the group consisting of:
1-propyl-8-(1-{[3-(trifluoromethyl)phenyl]-methyl}pyrazol-4-yl)-1,3,7-trihydropurine-2,6-dione;
1-propyl-8-[1-benzylpyrazol-4-yl]-1,3,7-trihydropurine-2,6-dione;
1-butyl-8-(1-{[3-fluorophenyl]methyl}pyrazol-4-yl)-1,3,7-trihydropurine-2,6-dione;
1-propyl-8-[1-(phenylethyl)pyrazol-4-yl]-1,3,7-trihydropurine-2,6-dione;
8-(1-{[5-(4-chlorophenyl)(1,2,4-oxadiazol-3-yl)]methyl}pyrazol-4-yl)-1-propyl-1,3,7-trihydropurine-2,6-dione;
8-(1-{[5-(4-chlorophenyl)(1,2,4-oxadiazol-3-yl)]methyl}pyrazol-4-yl)-1-butyl-1,3,7-trihydropurine-2,6-dione;
1,3-dipropyl-8-pyrazol-4-yl-1,3,7-trihydropurine-2,6-dione;
1-methyl-3-sec-butyl-8-pyrazol-4-yl-1,3,7-trihydropurine-2,6-dione;
1-cyclopropylmethyl-3-methyl-8-{1-[(3-trifluoromethylphenyl)methyl]pyrazol-4-yl}-1,3,7-trihydropurine-2,6-dione;
1,3-dimethyl-8-{1-[(3-fluorophenyl)methyl]pyrazol-4-yl}-1,3,7-trihydropurine-2,6-dione;
3-methyl-1-propyl-8-{1-[(3-trifluoromethylphenyl)methyl]pyrazol-4-yl}-1,3,7-trihydropurine-2,6-dione;
3-ethyl-1-propyl-8-{1-[(3-trifluoromethylphenyl)methyl]pyrazol-4-yl}-1,3,7-trihydropurine-2,6-dione;
1,3-dipropyl-8-(1-{[3-(trifluoromethyl)phenyl]methyl}pyrazol-4-yl)-1,3,7-trihydropurine-2,6-dione;
1,3-dipropyl-8-{1-[(3-fluorophenyl)methyl]pyrazol-4-yl}-1,3,7-trihydropurine-2,6-dione;
1-ethyl-3-methyl-8-{1-[(3-fluorophenyl)methyl]pyrazol-4-yl}-1,3,7-trihydropurine-2,6-dione;
1,3-dipropyl-8-{1-[(2-methoxyphenyl)methyl]pyrazol-4-yl}-1,3,7-trihydropurine-2,6-dione;
1,3-dipropyl-8-(1-{[3-(trifluoromethyl)-phenyl]ethyl}pyrazol-4-yl)-1,3,7-trihydropurine-2,6-dione;
1,3-dipropyl-8-{1-[(4-carboxyphenyl)methyl]pyrazol-4-yl}-1,3,7-trihydropurine-2,6-dione;
2-[4-(2,6-dioxo-1,3-dipropyl(1,3,7-trihydropurin-8-yl))pyrazolyl]-2-phenylacetic acid;
8-{4-[5-(2-methoxyphenyl)-[1,2,4]oxadiazol-3-ylmethoxy]phenyl}-1,3-dipropyl-1,3,7-trihydropurine-2,6-dione;
8-{4-[5-(3-methoxyphenyl)-[1,2,4]oxadiazol-3-ylmethoxy]phenyl}-1,3-dipropyl-1,3,7-trihydropurine-2,6-dione;
8-{4-[5-(4-fluorophenyl)-[1,2,4]oxadiazol-3-ylmethoxy]phenyl}-1,3-dipropyl-1,3,7-trihydropurine-2,6-dione:
1-(cyclopropylmethyl)-8-[1-(2-pyridylmethyl)pyrazol-4-yl]-1,3,7-trihydropurine-2,6-dione;
1-n-butyl-8-[1-(6-trifluoromethylpyridin-3-ylmethyl)pyrazol-4-yl]-1,3,7-trihydropurine-2,6-dione;
8-(1-{[3-(4-chlorophenyl)(1,2,4-oxadiazol-5-yl)]methyl}pyrazol-4-yl)-1,3-dipropyl-1,3,7-trihydropurine-2,6-dione;
1,3-dipropyl-8-[1-({5-[4-(trifluoromethyl)phenyl]isoxazol-3-yl}methyl)pyrazol-4-yl]-1,3,7-trihydropurine-2,6-dione;
1,3-dipropyl-8-[1-(2-pyridylmethyl)pyrazol-4-yl]-1,3,7-trihydropurine-2,6-dione;
3-{[4-(2,6-dioxo-1,3-dipropyl-1,3,7-trihydropurin-8-yl)pyrazolyl]methyl}benzoic acid;
1,3-dipropyl-8-(1-{[6-(trifluoromethyl)(3-pyridyl)]methyl}pyrazol-4-yl)-1,3,7-trihydropurine-2,6-dione;
1,3-dipropyl-8-{1-[(3-(1H-1,2,3,4-tetraazol-5-yl)phenyl)methyl]pyrazol-4-yl}-1,3,7-trihydropurine-2,6-dione;
6-{[4-(2,6-dioxo-1,3-dipropyl-1,3,7-trihydropurin-8-yl)pyrazolyl]methyl}pyridine-2-carboxylic acid;
3-ethyl-1-propyl-8-[1-(2-pyridylmethyl)pyrazol-4-yl]-1,3,7-trihydropurine-2,6-dione;
8-(1-{[5-(4-chlorophenyl)isoxazol-3-yl]methyl}pyrazol-4-yl)-3-ethyl-1-propyl-1,3,7-trihydropurine-2,6-dione;
8-(1-{[3-(4-chlorophenyl)(1,2,4-oxadiazol-5-yl)]methyl}pyrazol-4-yl)-3-ethyl-1-propyl-1,3,7-trihydropurine-2,6-dione;
3-ethyl-1-propyl-8-(1-{[6-(trifluoromethyl)(3-pyridyl)]methyl}pyrazol-4-yl)-1,3,7-trihydropurine-2,6-dione;
1-(cyclopropylmethyl)-3-ethyl-8-(1-{[6-(trifluoromethyl)(3-pyridyl)]methyl}pyrazol-4-yl)-1,3,7-trihydropurine-2,6-dione; and
3-ethyl-1-(2-methylpropyl)-8-(1-{[6-(trifluoromethyl)(3-pyridyl)]methyl}pyrazol-4-yl)-1,3,7-trihydropurine-2,6-dione
or a pharmaceutically acceptable salt, tautomer, isomer, a mixture of isomers, or prodrug thereof.

30. The method of claim 1, wherein the A_{2B} receptor antagonist is a compound of the formula: 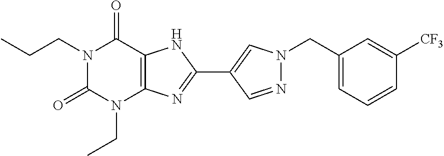
or a pharmaceutically acceptable salt, tautomer, isomer, a mixture of isomers, or prodrug thereof.

31. The method of claim 1, wherein the A_{2B} receptor antagonist is a prodrug of Formula III having the formula: 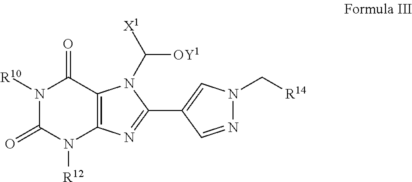
wherein:
R¹⁰ and R¹² are independently lower alkyl;
R¹⁴ is optionally substituted phenyl;
X¹ is hydrogen or methyl; and
Y¹ is —C(O)R¹⁷, in which R¹⁷ is independently optionally substituted lower alkyl, optionally substituted aryl, or optionally substituted heteroaryl; or
Y¹ is —P(O)(OR¹⁵)₂, in which R¹⁵ is hydrogen or lower alkyl optionally substituted by phenyl or heteroaryl;
and the pharmaceutically acceptable salts thereof.

32. The method of claim 31, wherein the compound is selected from the group consisting of
[3-ethyl-2,6-dioxo-1-propyl-8-(1-{[3-(trifluoromethyl)phenyl]methyl}pyrazol-4-yl)-1,3,7-trihydropurin-7-yl]methyl acetate;
[3-ethyl-2,6-dioxo-1-propyl-8-(1-{[3-(trifluoromethyl)phenyl]methyl}pyrazol-4-yl)-1,3,7-trihydropurin-7-yl]methyl 2,2-dimethylpropanoate;
[3-ethyl-2,6-dioxo-1-propyl-8-(1-{[3-(trifluoromethyl)phenyl]methyl}pyrazol-4-yl)-1,3,7-trihydropurin-7-yl]methyl butanoate; and
[3-ethyl-2,6-dioxo-1-propyl-8-(1-{[3-(trifluoromethyl)phenyl]methyl}-pyrazol-4-yl)(1,3,7-trihydropurin-7-yl)]methyl dihydrogen phosphate.

33. The method of claim 1, further comprising administering an additional therapeutic agent selected from the group consisting of cardiac glycosides, vasodilators/calcium channel blockers, prostacyclins, anticoagulants, diuretics, endothelin receptor blockers, phosphodiesterase type 5 inhibitors, nitric oxide inhalation, arginine supplementation and combinations thereof.

34. The method of claim 33, wherein the additional agent is an endothelin receptor blocker.

35. The method of claim 34, wherein the endothelin receptor blocker is ambrisentan.

36. The method of claim 35, wherein the additional agent is administered simultaneously or sequentially with the A_{2B} adenosine receptor antagonist.

37. A method of inhibiting overexpression of a collagen, an extracellular matrix protein, and/or an extracellular matrix enzyme in a pulmonary arterial smooth muscle cell which method comprises contacting the cell with an effective amount of an A_{2B} adenosine receptor antagonist.

38. The method of claim 37, wherein the collagen, the extracellular matrix protein, and/or the extracellular matrix enzyme is selected from ADAMTS1, ADAMTS8, CDH1, MMPI, MMP12, HAS1, ITGA7, COL1A1, COL8A1 or CTGF.

39. A method of reducing IL-6, IL-8, G-CSF, and/or thromboxane expression in a pulmonary arterial smooth muscle cell which method comprises contacting the cell with an effective amount of an A_{2B} adenosine receptor antagonist.

40. A method of reducing IL-8 and/or ET-1 expression in a pulmonary arterial endothelial cell which method comprises contacting the cell with an effective amount of an A_{2B} adenosine receptor antagonist.

41. A method of inhibiting proliferation or migration of a pulmonary arterial smooth muscle cell which method comprises contacting the cell with an effective amount of an A_{2B} adenosine receptor antagonist.

42. A method of inhibiting vascular wall thickening in a patient in need thereof, which comprises administering to the patient a therapeutically effective amount of an A_{2B} adenosine receptor antagonist.

43. A method of decreasing right ventricular systolic pressure (RVSP) and/or right ventricular hypertrophy in a patient in need thereof, which comprises administering to the patient a therapeutically effective amount of an A_{2B} adenosine receptor antagonist.

44. A method of improving lung function in a patient in need thereof, which comprises administering to the patient a therapeutically effective amount of an A_{2B} adenosine receptor antagonist.
